(19) United States
(12) Patent Application Publication
(10) Pub. No.: US 2025/0144354 A1
(43) Pub. Date: May 8, 2025

(54) POSITIVE PRESSURE BREATHING CIRCUIT

(71) Applicant: Fisher & Paykel Healthcare Limited, Auckland (NZ)

(72) Inventors: Brett John HUDDART, Auckland (NZ); Peter Lawrence GRYLLS, Auckland (NZ); David Robin WHITING, Auckland (NZ); Andrew Gordon GERRED, Auckland (NZ); Andrew Paul Maxwell SALMON, Auckland (NZ); Douglas Richard WRIGHT, Auckland (NZ); Ian Patrick Sarsfield HICKEY, Auckland (NZ); Daniel John SMITH, Auckland (NZ); Mark REEVES, Auckland (NZ); Jonathan David HARWOOD, Auckland (NZ); Lucas Michael TOOVEY, Auckland (NZ); David John LOVE, Durban North (ZA)

(21) Appl. No.: 18/837,328

(22) PCT Filed: Feb. 11, 2022
(86) PCT No.: PCT/NZ2022/050019

§ 371 Date: Aug. 9, 2024

(51) Int. Cl. A61M 16/08; A61M 16/00; A61M 16/06; A61M 16/10; A61M 16/20

(52) U.S. Cl. CPC A61M 16/0883; A61M 16/0003; A61M 16/0069; A61M 16/0627; A61M 16/0875; A61M 16/10; A61M 16/209; A61M 2016/0027; A61M 2016/0039; A61M 2016/0042; A61M 2202/0007; A61M 2202/0208; A61M 2202/0241; A61M 2202/025; A61M 2202/0275; A61M 2205/103; A61M 2205/3303; A61M 2205/3327; A61M 2205/3334; A61M 2205/3344; A61M 2205/3379; A61M 2230/005; A61M 2230/40; A61M 2240/00

(57) ABSTRACT

The present disclosure relates to a positive pressure breathing circuit and a method for ventilating a patient. The breathing circuit can be used in any type of pressurized breathing therapy including, for example, continuous positive air (way) pressure (CPAP) therapy and bilevel positive air pressure therapy where the inspiratory and expiratory pressures differ.

### FIELD

The present disclosure relates to a positive pressure breathing circuit and a method for ventilating a patient. The breathing circuit can be used in any type of pressurized breathing therapy including, for example, continuous positive air (way) pressure (CPAP) therapy and bilevel positive air pressure therapy where the inspiratory and expiratory pressures differ.

### BACKGROUND

Breathing circuits can help a patient to breath by opening up their airways and/or supplying specific breathing gases for a particular medicinal purpose. The breathing gases may be supplied at a flow rate that is higher than an average inspiratory flow rate to ensure there is no shortage of breathing gases. In the case of CPAP therapy, the flow supplied to the patient is usually higher than the peak inspiratory flow, rather than the average inspiratory flow.

Some traditional breathing circuits use a mixed breathing gas including a blend of air and oxygen gas that is supplied to a patient via an inspiratory tube. The required oxygen saturation levels in the patient's blood can be achieved by adjusting the ratio of the oxygen in the oxygen/air blend. However, a problem with this breathing circuit is the positive pressure experienced by the patient is the result of a continuous supply of the mixed breathing gas during both inhalation and exhalation, which results in a significant wastage of the oxygen gas. There is therefore a need for an improved breathing circuit.

### SUMMARY

An embodiment relates to a positive pressure breathing circuit for ventilating a patient, the breathing circuit including:
an inspiratory member that is connectable to: i) a patient interface for supplying a breathing gas, ii) a first source of a pressurized first gas and iii) a second source of a pressurized second gas, wherein the inspiratory member includes a first non-return valve and the first gas enters the inspiratory member upstream of the first non-return valve and the second gas enters the inspiratory member downstream of the first non-return valve; and
a pressure regulation device configured to regulate pressure in the breathing circuit, including venting exhaled gas.

An embodiment relates to a positive pressure breathing circuit for ventilating a patient, the breathing circuit including:
an inspiratory member including a proximal portion that is connectable to a patient interface for supplying a breathing gas, and a distal portion that is connectable to a first source of a pressurized first gas and a second source of a pressurized second gas, wherein the second gas enters the inspiratory member downstream to where the first gas enters the inspiratory member; and
a pressure regulation device configured to regulate pressure in the breathing circuit, including venting exhaled gas.

An advantage provided by the second gas entering the inspiratory member distally is that the inspiratory member has few or no additional components proximal to the patient interface making the breathing circuit lighter which makes the patient interface more comfortable than if the second gas entered the inspiratory member closer to the patient interface. The inspiratory member may include an inspiratory tube.

The breathing circuit may include an expiratory member which receives the exhaled gas from the patient interface.

The expiratory member may include an expiratory tube extending away from the patient interface. In one example, the expiratory member may be connected to the patient interface. In another example, proximal portions of the expiratory member and the inspiratory member may be connected adjacent to the patient interface.

The expiratory member is configured so that the excess supply of the first gas in the expiratory member downstream of the second non-return valve and the exhaled gas in the expiratory member downstream of the second non-return valve are vented from the breathing circuit

The inspiratory member may include a first non-return valve and the first gas enters the inspiratory member upstream of the first non-return valve and the second gas enters the inspiratory member downstream of the first non-return valve. That is to say, the first non-return valve is located between the first gas and the second gas entering the inspiratory member.

Throughout this specification, the first non-return valve inhibits the exhaled gas from passing upstream of the first non-return valve. The first non-return valve inhibits the exhaled gas from passing upstream of the first non-return valve, but this does not necessarily mean that the first non-return valve completely blocks the flow. The first non-return valve may of course block the flow.

In addition, by locating the first non-return valve distally, the inspiratory member has few or no additional components proximal to the patient interface, allowing the distal portion of the breathing circuit attached to the patient interface to be lighter than if the first non-return valve was proximal to the patient interface and the second gas entered the inspiratory member closer to the patient interface.

The first non-return valve is configured to inhibit the second gas flowing upstream toward the first gas entering the inspiratory member.

The inspiratory member is configured so that a volume of the second gas can enter and flow toward the patient interface during patient exhalation and be stored in the inspiratory member.

The expiratory member may have a second non-return valve to inhibit the exhaled gas from re-entering the patient interface.

Throughout this specification, the second non-return valve inhibits the flow of the excess supply of the first gas passing upstream of the second non-return valve, but this does not necessarily mean that the second non-return valve completely blocks the flow. The second non-return valve may of course block the flow.

The internal volume of the inspiratory member for receiving the volume of the second gas can be changed by changing the length of the inspiratory member to accommodate a desired volume of the second gas during patient exhalation. For example, the inspiratory member may be expandible and contractible in an axial direction of the tube to change the length of the member. In another example, the breathing circuit may have a set of the inspiratory members of different internal volume, and the member of desired internal volume can be chosen from the set. In yet another example, the inspiratory member may have a set of markings thereon signifying the internal volume at particular lengths. In use, a user may select the desired internal volume by cutting the inspiratory member at one of the markings.

The internal volume may be changed by changing the diameter of the inspiratory member. For example, the inspiratory member may have an expandable diameter over part or all of the length of the inspiratory member.

The pressure regulation device may be configured to regulate pressure in the breathing circuit.

In one example, the pressure regulation device may be directly connected to the patient interface. For example, the pressure regulation device may be integrally formed with the patient interface, connected to an outlet port of the patient interface, or connected to the patient interface, for example, by a connector such as Y-piece. In the situation where the pressure regulation device is on the patient interface, there may be no need for an expiratory member.

In another example, the pressure regulation device may be connected to the expiratory member and vent the exhaled gas from the expiratory member. In one example, the pressure regulation device may be connected to an end of the expiratory member. In this example, the expiratory member is not connected to the inspiratory member.

For instance, the pressure regulation device may include a pressure relief valve, such as a positive end expiratory pressure valve (expiratory tube PEEP valve) for venting exhaled gas.

In another example, the pressure regulation device may be configured to regulate pressure in the inspiratory member. For instance, the pressure regulation device in the inspiratory member may include a pressure relief valve, such as a positive end expiratory pressure valve (inspiratory member PEEP valve) for venting the first gas supplied in excess to the breathing circuit.

In another example, the pressure regulation device may include a first control valve for controlling the pressure of the first gas supplied to the breathing circuit.

In another example, the pressure regulation device may include a second control valve for controlling the pressure of the second gas supplied to the breathing circuit.

The inspiratory member may be connectable to the expiratory member so that any excess of the first gas supplied to the inspiratory member passes (from the inspiratory member) to the expiratory member without passing through the patient interface. That is to say the inspiratory member and the expiratory member are connected in a loop configuration and the excess supply of the first gas is conveyed from the inspiratory member to the expiratory member in the loop configuration remote from the patent interface.

The distal portion of the expiratory member and the distal portion of the inspiratory member may be connected to form the loop configuration. That is to say, they are connected remotely from the patent interface.

The expiratory member may include a second non-return valve to inhibit the first gas from entering the patient interface from the expiratory member.

The expiratory member is configured so that the first gas and the exhaled gas in the expiratory member downstream of the second non-return valve are vented from the breathing circuit. Specifically, the expiratory member is configured to vent the first gas supplied in excess to the breathing circuit that flows to the expiratory member and the exhaled gas received by the expiratory member.

The second non-return valve also inhibits the exhaled gas from being rebreathed.

The expiratory member may have a substantially constant volume. That is to say in one example, the expiratory member may not have a volume changing structure volume such as a bellows, collapsible chamber, or flexible walled passage or alike. The volume of the expiratory member may fluctuate by a small amount due to pressure changes, but the macro structure of the expiratory member is not configured to change with changes in pressure.

The expiratory member may have a constant volume upstream of the second non-return valve.

The expiratory member may have a constant volume downstream of the second non-return valve.

The inspiratory member may be connected to the expiratory member downstream of the second non-return valve.

In one example, the breathing circuit comprises a bypass member interconnecting the inspiratory member and the expiratory member that conveys the excess supply of the first gas from the inspiratory member to the expiratory member.

The bypass member may include a bypass tube.

The bypass member may interconnect distal portions of the inspiratory and expiratory members.

The bypass member may connect to the expiratory member downstream of the second non-return valve.

In another example, the inspiratory member and the expiratory member are directly interconnected.

The inspiratory member and the expiratory member may have a continuous open line so the first gas can pass from the inspiratory member to the expiratory member in one direction.

The first gas received by the expiratory member is vented from the breathing circuit unobstructed, and the expiratory member is configured so that the exhaled gas passes through the second non-return valve and is vented from the breathing circuit.

The expiratory member may be configured so that the excess supply of the first gas and the exhaled gas downstream of the second non-return valve are vented from the breathing circuit without re-entering the inspiratory member. This can be achieved by the first gas and the second gas being supplied to the inspiratory member, and the patient exhaling the exhaled gas at an exhaled pressure.

The pressure regulation device may include a positive end expiratory pressure valve (PEEP valve) on the distal portion of the expiratory member.

The positive end expiratory pressure valve of the expiratory member may have a pressure setting ranging from about 2.5 to 35.0 cmH₂O, about 4.5.0 to 25.0 cmH₂O, about 6.5 to 15 cmH₂O, or about 8.0 to 12.0 cmH₂O, or about 10.0 cmH₂O.

The pressure regulation device of the expiratory member may have a higher pressure setting than the pressure regulation device of inspiratory member. The difference in pressure may inhibit flow of the breathing gas from the inspiratory member to the expiratory member other than that caused by the patient.

The pressure regulation devices of at least one of the expiratory member and the inspiratory member may be a positive end expiratory pressure valve (PEEP valve).

The pressure regulation devices of at least one of the expiratory member and the inspiratory member may be a restriction orifice.

The pressure regulation device may include an over pressure relief valve that is intended to release the pressure from the breathing circuit. The over pressure relief valve may have a higher setting than the pressure relief valve of the expiratory member, and if present, the pressure relief valve of the inspiratory member. The over pressure relief valve may be located on the inspiratory member. The over pressure relief valve may be located on the expiratory member. If the bypass member interconnecting the inspiratory and expiratory members is present, the over pressure relief valve may be located on the bypass member. The over pressure relief valve may be located on any one or a combination of the inspiratory member, the expiratory member and the bypass member.

The breathing circuit may include a humidification device for humidifying part of, or all of, the breathing gas. For example, the humidification device may be humidifying the first gas. In this instance, the humidification device may be located upstream of the second gas entering the inspiratory member, such as upstream of the first non-return valve.

In another example, the humidification device may humidify the first gas and the second gas. In this instance, the humidification device will be located downstream of the second gas entering the inspiratory member. In this position, the humidification device would be located downstream of the first non-return valve if present. An advantage in this arrangement is that the first non-return valve is kept dry which is better for achieving reliable functionality. In addition, any secretions from the patient are unlikely to contact the first non-return valve due to its position upstream of the second gas entering the inspiratory member.

The humidification device may have a humidification chamber in which the water and the breathing gas contact, and the chamber has a volume in which the second gas can accumulate. Especially when the patient is exhaling. This further increases the likelihood of the patient inhaling the second gas at the start of their breath.

As the second gas can be stored in the humidifier device while the patient is exhaling in addition to the inspiratory member, the volume of the humidification chamber should be considered when equating the tidal volume to the inspiratory member.

The second gas may be supplied at a constant flow rate.

The first gas may be supplied at a constant flow rate.

The breathing circuit may include the first source of the first gas in which the first source includes a variable flow generator that is operable for supplying a high pressure during patient inhalation and a low pressure during patient exhalation for the first gas. The variable flow generator may also be operable to provide constant pressure. That is to say, the inspiratory pressure or the IPAP is higher, relative to the expiratory pressure or the EPAP which is lower. The flow generator may cycle between the high pressure during patient inhalation and the low pressure during patient exhalation during continuous patient breathing. For example, the variable flow generator may be a non-invasive ventilation or a PAP device that has a variable speed blower. The variable flow generator may also supply the first gas at a constant positive air pressure during patient inhalation and patient exhalation.

The breathing circuit may include a sensor for detecting when a patient inhales and/or exhales. Typically, the gas flow generator operates at the high pressure when the patient inhales, and at the low pressure when the patient exhales. In the situation in which the first gas is air, the variable speed blower may include an inspiratory positive airway pressure (IPAP) and an expiratory positive airway pressure (EPAP).

The sensor may be a flow sensor for detecting the flow of the first gas in the breathing circuit. The flow sensor may be located within the flow generator upstream or downstream of a blower of the flow generator. For example, the flow sensor may be located at the inlet or outlet of the flow generator. In another example, the flow sensor may be located at the inspiratory member to detect when the patient inhales. An output from the flow sensor can be used to determine when the patient starts and/or ends inhaling, and can be used to operate the flow generator in high pressure flow and low pressure, such as between IPAP or EPAP.

In another example, the flow sensor may be located in the expiratory member to detect when the patient exhales. An output from the flow sensor can be used to determine when the patient starts and/or ends exhaling, and can be used to operate the pressure regulation device in high pressure flow and low pressure, such as between IPAP or EPAP.

The sensor may include a pressure sensor for detecting the pressure of the first gas in the breathing circuit downstream of the flow generator. In one example, the pressure sensor may be located on the inspiratory member downstream of the flow generator and upstream of the second gas entering the inspiratory member. In another example, the pressure sensor may be within the flow generator and downstream of a blower of the flow generator. In yet another example, the pressure sensor may be located at the patient interface.

When the breathing circuit includes a variable flow generator, the pressure regulation device of the breathing circuit may, in one example, include a pressure relief valve, such as a PEEP valve. The pressure relief valve may be located in the expiratory member for venting the exhaled gas.

In another example, the pressure regulation device may include a restriction orifice having an aperture of fixed opening size.

In another example, the pressure regulation may include a constant flow valve

In further examples, the internal volume may range from about 315 ml to 760 ml for adult patients, or range from about 400 to 600 ml. For pediatric patients, the internal volume may range from about 100 ml to 450 ml, or range from about 200 to 400 ml. For neonatal patients, the internal volume may range from about 50 to 200 ml, or range from about 100 to 150 ml.

The pressure of exhaled gas in the expiratory member may be greater than the pressure of the breathing gas in inspiratory member.

The first gas may be pressurized air. The first gas may be pressurized air enriched with oxygen.

In one example, the second gas may be pressurized oxygen gas.

In another example, the second gas may be a pressurized gas including one or any combination of: oxygen gas, heliox, or an anaesthetic gas. The anaesthetic gas could be nitrous oxide or a 50:50 mixture of nitrous oxide and oxygen gas.

Pressurized oxygen gas may be supplied from a liquified oxygen source, a bottled oxygen source or from an oxygen concentrator source.

The breathing circuit may include a patient interface. The patient interface may be a sealed patient interface. For example, the patient interface includes either one or any combination of a full-face mask (also known as an oro-nasal mask), a sealed nasal cannula, a sealed oral mask, a sealed nasal mask, a nasal pillows interface, or a tracheostomy member. The first non-return valve may be arranged on the patient interface.

The patient interface may have an inlet connection that connects to the inspiratory member, and an outlet connection that connects to the expiratory member.

The patient interface may have a coupling to which a Y-piece, is or can be connected, in which one leg of the Y-piece is an inlet connection that connects to the inspiratory member, and another leg is an outlet connection that connects to the expiratory member.

The positive end expiratory pressure valve of the expiratory member may be fitted directly to the outlet connection of the Y-piece.

The inspiratory member may be directly connected to the patient interface either with or without a Y-piece. That is to say, there may be no intervening operations such as humidifiers, heat and moisture exchangers or other items that have the potential to increase dead space in the breathing circuit between the inspiratory member and the patient interface.

An embodiment relates to a positive pressure breathing circuit for ventilating a patient, the breathing circuit including:
an inspiratory member including a proximal portion that is connectable to a patient interface for supplying a breathing gas, and a distal portion that is connectable to a source of a pressurized first gas, wherein the inspiratory member is connectable to a pressurized second gas that enters the inspiratory member downstream of a position to where the first gas enters the inspiratory member; and
a flow generator for supplying the first gas at a controlled pressure to the inspiratory member.

The breathing circuit may include a pressure regulation device configured to regulate pressure in the breathing circuit, including venting exhaled gas.

The breathing circuit may have an expiratory member configured to vent the exhaled gas from the patient interface.

In one example, the flow generator may be a variable flow generator that is operable to provide high pressure during patient inhalation and low pressure during patient exhalation. The variable flow generator may also be operable to provide constant pressure. That is to say, the inspiratory pressure or the IPAP is higher, relative to the expiratory pressure or the EPAP which is lower.

One of the advantages of this embodiment is that variable flow generator is operable to supply the first gas at set pressures, and in turn, controls the total pressure in the breathing circuit. This may include the option of the inspiratory member not having a separate venting device. In this instance, the inspiratory member may be closed, that is without a vent.

In another example, the flow generator may be a constant flow generator. That is the flow generator can be used for CPAP, which delivers a constant pressure for a given setting. It will be appreciated that a user may change the setting as desired.

The breathing circuit may have a pressure regulation device for regulating the pressure in the expiratory member.

The pressure regulation device for regulating the pressure in the expiratory member may be set to slightly higher pressure than the target pressure of the variable flow generator. For example 0.5 to 2.0 cmH₂0 higher than the target pressure of the flow generator. This inhibits the second gas continuously flowing through the regulation device, which will then vent when the patient exhales.

The inspiratory member may be configured so that the second gas enters a proximal portion of the inspiratory member. In this situation, the inspiratory member may include a first non-return valve downstream of the second gas entering the inspiratory member.

In this situation in which the second gas enters the proximal portion, the breathing circuit may include a humidification device for humidifying the second gas prior to entering the inspiratory member.

The inspiratory member may be configured so that the second gas enters a distal portion of the inspiratory member. In this situation, the inspiratory member may include a first non-return valve upstream of the second gas entering the inspiratory member. That is to say, the first non-return valve may be located between the first gas and the second gas entering the inspiratory member.

Elements of the breathing circuit may be connected together or pre-assembled into a module. The module may be connected to other elements by a user to complete the breathing circuit, or two or more modules may be connected together, which in turn may form the breathing circuit or be connected to other elements. Examples of possible modules may include any one or a combination of the following:
Module 1: The inspiratory member and the expiratory member interconnect at proximal portions thereof.
Module 2: The inspiratory member and the expiratory member interconnect at proximal portions thereof, and the pressure regulation device in the expiratory member.
Module 3: The inspiratory member and the expiratory member interconnect together at the distal portions thereof, the pressure regulation device in the expiratory member, and the second gas inlet port in the inspiratory member.
Module 4: The inspiratory member and the expiratory member interconnect at proximal portions thereof, the pressure regulation device in the expiratory member, the second gas inlet, and the first non-return valve.
Module 5: The inspiratory member and the expiratory member interconnect at distal portions thereof, in which the inspiratory member includes the first gas inlet port, the second gas inlet port, and the first non-return valve, the bypass member interconnecting distal portion of the inspiratory and expiratory members; and the pressure regulation device.
Module 6: The same as Module 5 with the addition of the second non-return valve in the expiratory member.
Module 7: The same as any one of Modules 1 to 6 with the patient interface coupling for connecting to a patent interface.
Module 8: The same as any one of Modules 1 to 7 including the patient interface connected to the proximal portions.
Module 9: The variable flow regulator including the flow sensor and the pressure sensor.
Module 10: The inspiratory member having the first gas inlet port, the second gas inlet port, and the first gas non-return valve.
Module 11: The variable flow regulator including the flow sensor and the pressure sensor, and the distal portion of the inspiratory member including the first non-return valve and the second gas inlet port.
Module 12: The variable flow regulator including the flow sensor and the pressure sensor, distal portions of the inspiratory member and the expiratory member that are interconnected, the first gas inlet port in the inspiratory member, the first non-return valve, the second gas inlet port; and the pressure regulation device for regulating the pressure in the expiratory member.
Module 13: The same as Module 12 with the addition of the second non-return valve in the expiratory member.

An embodiment relates to a method for ventilating a patient, the method including:
providing a positive pressure breathing circuit having:
   an inspiratory member that is connectable to: i) a patient interface for supplying a breathing gas, ii) and a first source of a pressurized first gas and iii) a second source of a pressurized second gas, wherein the inspiratory member includes a first non-return valve and the first gas enters the inspiratory member upstream of the first non-return valve and the second gas enters the inspiratory member downstream of the first non-return valve; and
   a pressure regulation device configurated to regulate pressure in the breathing circuit, including venting exhaled gas; and
supplying the first gas and the second gas into the distal portion of the inspiratory member, wherein the second gas enters the inspiratory member downstream to where the first gas enters the inspiratory member.

An embodiment relates to a method for ventilating a patient, the method including:
providing a positive pressure breathing circuit having:
   an inspiratory member including a proximal portion that is connectable to a patient interface for supplying a breathing gas, and a distal portion that is connectable to a first source of a pressurized first gas and a second source of a pressurized second gas; and
   a pressure regulation device configurated to regulate pressure in the breathing circuit, including venting exhaled gas; and
supplying the first gas and the second gas into the distal portion of the inspiratory member, wherein the second gas enters the inspiratory member downstream to where the first gas enters the inspiratory member.

The positive pressure breathing circuit may include any one or a combination of the features of the breathing circuit described herein. For example, the breathing circuit provided may include an expiratory member configured to vent exhaled gas from the patient interface.

The method may include selecting an internal volume of the inspiratory member in which the second gas can be stored. Selecting the internal volume may include adjusting the volume so that a therapeutic amount of the second gas can be stored in the inspiratory member. Selecting the internal volume may include determining where to severe the inspiratory member based on volume markings spaced along the length of the member. The method may include adjusting the internal volume by severing the inspiratory member at one of the markings.

The method may include a step of regulating the pressure in the breathing circuit which may include venting exhaled gas from the expiratory member. The venting may be achieved using a restriction device, such as a pressure relief valve, a PEEP valve, an orifice, and constant flow device that maintains a constant flow irrespective of the pressure differential across the device.

In the situation in which the distal portions of the inspiratory member and the expiratory member are interconnected to form a loop configuration, the method may include the first gas being supplied to the inspiratory member and any excess supply of the first gas is conveyed from the inspiratory member to the expiratory member by the interconnection of the inspiratory member and the expiratory member without passing through the patient interface.

The breathing circuit provided may be configured with a bypass member interconnecting the inspiratory member and the expiratory member. In this situation, the step of regulating the pressure may include conveying the excess supply of the first gas from the inspiratory member to the expiratory member via the bypass member.

The method may include releasing overpressure from the circuit using an overpressure relief valve.

The method may include humidifying the breathing gas. For example, humidifying the first and the second gas.

The second gas may be supplied at a substantially constant flow rate.

In one example, the first gas may be supplied at a constant flow rate.

In another example, the first gas may be supplied at a variable flow rate. For example, the method may include operating a variable flow generator to supply the first gas at a high pressure flow during patient inhalation and a low pressure flow during patient exhalation, such as between IPAP or EPAP.

The step of operating the variable flow generator may include sensing flow in the breathing circuit and using output data of a sensor sensing the flow to operate the variable flow generator. The output data of the sensor may respond to when the patient inhales and/or exhales. The sensor may include a flow sensor, for example, in the expiratory member. The sensor may include a pressure sensor, for example, in the expiratory member.

An embodiment relates to a method for ventilating a patient, the method including:
providing a positive pressure breathing circuit including:
   an inspiratory member including a proximal portion that is connectable to a patient interface for supplying a breathing gas, and a distal portion that is connectable to a source of a pressurized first gas, wherein the inspiratory member is connectable to a pressurized second gas that enters the inspiratory member downstream of a position to where the first gas enters the inspiratory member; and
   a flow generator for supplying the first gas, and
operating the flow generator at a controlled pressure to the inspiratory member.

The step of providing a positive pressure breathing circuit including providing a pressure regulation device configured to regulate pressure in the breathing circuit, including venting exhaled gas

The embodiments described in the paragraphs [0004], [0005], [0091], [0102], [0103], [0104] and [0116] may include any one or a combination of the features described herein.

Throughout this specification the term “excess supply of the first gas”, or variations thereof, refers to an amount of the first gas supplied by the flow generator that is not delivered to the patient interface.

The components of the breathing circuit described herein, including the inspiratory tube and the expiratory tube may be made of any suitable medical grade materials, including flexible plastic tubing that is substantially non-stretchable. Moreover, suitably the inspiratory and the expiratory tubes meet the ISO-5367 standard for compliance.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 is a schematic illustration of a breathing circuit including an inspiratory member and an expiratory member that are connected at proximal and distal portions to provide a loop configuration, and in which first gas and second gas sources are connected to a distal portion of the inspiratory member.

FIG. 2 is a schematic illustration of a breathing circuit including an inspiratory member and an expiratory member that are connected at proximal portions, in which a first gas source is connected to a distal portion and a second gas source is connected distally to the proximal portion, that is, either in a middle portion or at a distal portion of the inspiratory member, and an overpressure release valve.

FIG. 3 is a schematic illustration of the breathing circuit shown in FIG. 1 with proportional control valves for controlling the flow of the first and second gas sources.

FIG. 4 is a schematic illustration of the breathing circuit shown in FIG. 2 including a humidifier.

FIG. 5 is a schematic illustration of the breathing circuit shown in FIG. 3 including a humidifier.

FIG. 6 is a schematic illustration of the breathing circuit shown in FIG. 1 in which distal and proximal portions of the inspiratory and expiratory members are interconnected and a pressure regulation device includes a flow restrictor in the expiratory tube.

FIG. 7 is a schematic illustration of a breathing circuit including an inspiratory member and an expiratory member that are connected at proximal portions, a first gas source connected to a distal portion of the inspiratory member, in which the first gas source includes a variable flow generator which also provides the function of a pressure regulation device for the breathing circuit.

FIG. 8 is a schematic illustration of a breathing circuit in which a first gas source including a variable flow generator and a second gas source are connected to a distal portion of an inspiratory member.

FIG. 9 is a schematic illustration of the breathing circuit shown in FIG. 8 in which the second gas source is connected to a proximal portion of the inspiratory member and upstream of a non-return valve.

FIG. 10 is a schematic illustration of the breathing circuit shown in FIG. 8 in which a flow restriction device is provided at the distal interconnection between the inspiratory and expiratory members.

FIG. 11 is a schematic illustration of the breathing circuit shown in FIG. 7 in which components within the box shown in dashed lines may be preassembled as a module.

FIG. 12 is a schematic illustration of the breathing circuit shown in FIG. 8 in which components within each box shown in dashed lines may be preassembled as a module.

FIG. 13 is a schematic illustration of the breathing circuit shown in FIG. 10 in which components within each box shown in dashed lines may be preassembled as a module.

FIG. 14 is a schematic illustration of the breathing circuit shown in FIG. 8 in which components within the box shown in dashed lines may be preassembled as a module.

FIG. 15 is a schematic illustration of the breathing circuit shown in FIG. 9 in which components within the box shown in dashed lines may be preassembled as a module.

FIG. 16 is a schematic illustration of a breathing circuit in which a first gas source includes a first (inlet) variable flow generator connected to a distal portion of the inspiratory member, a second gas source is connected to a distal portion of the inspiratory member, and a pressure regulation device includes a second (outlet) variable flow generator that provides back pressure to an expiratory member.

FIG. 17 is a schematic illustration of a breathing circuit in which a first gas source includes a first variable flow generator connected to a distal portion of the inspiratory member, a second gas source is connected to a proximal portion of the inspiratory member, and a pressure regulation device includes a second variable flow generator that provides back pressure to an expiratory member.

FIG. 18 is a schematic illustration of a breathing circuit including an inspiratory member and an expiratory member that are connected at proximal portions, a first gas source connected to a distal portion of the inspiratory member, a second gas source connected to a proximal portion at a point upstream of a non-return valve, and an overpressure valve.

FIG. 19 is a block diagram illustrating method steps for ventilating a patient.

### DETAILED DESCRIPTION

An embodiment will now be described in the following text which includes reference numerals that correspond to features illustrated in the accompanying Figures. To maintain clarity of the Figures, however, not all reference numerals are included in each Figure. Although certain examples are described herein, those of skill in the art will appreciate that the disclosure extends beyond the specifically disclosed examples and/or uses manually connected to a connection port of the interface 21 or a length of the tubing can interconnect the tube connector and a connection portion of the patient interface 21. In another example, not illustrated in the Figures, the inspiratory tube 11 and the expiratory tube 12 may be directly connected to an inlet connection of the patient interface 21 and an outlet connection of the patient interface 21 respectively.

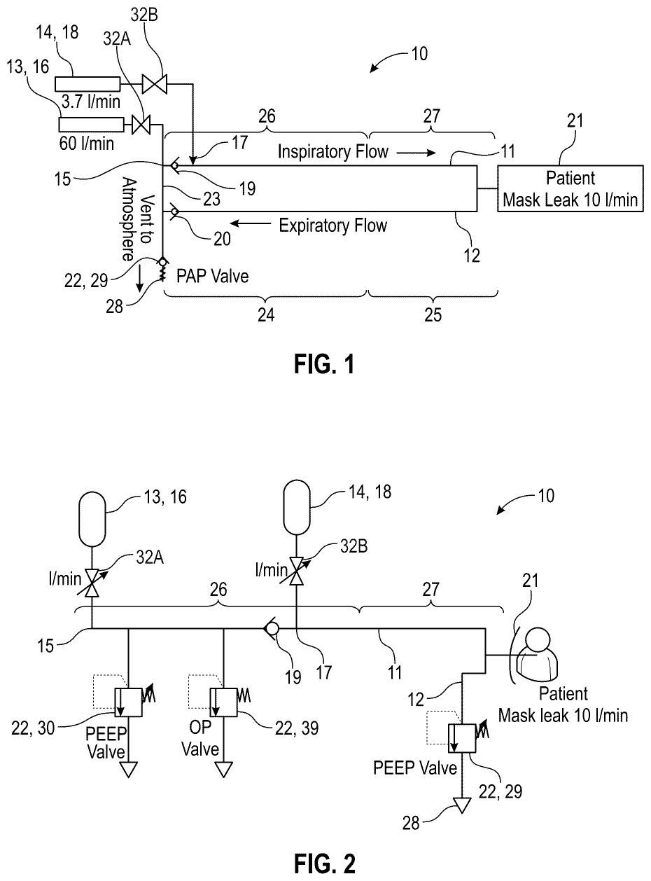

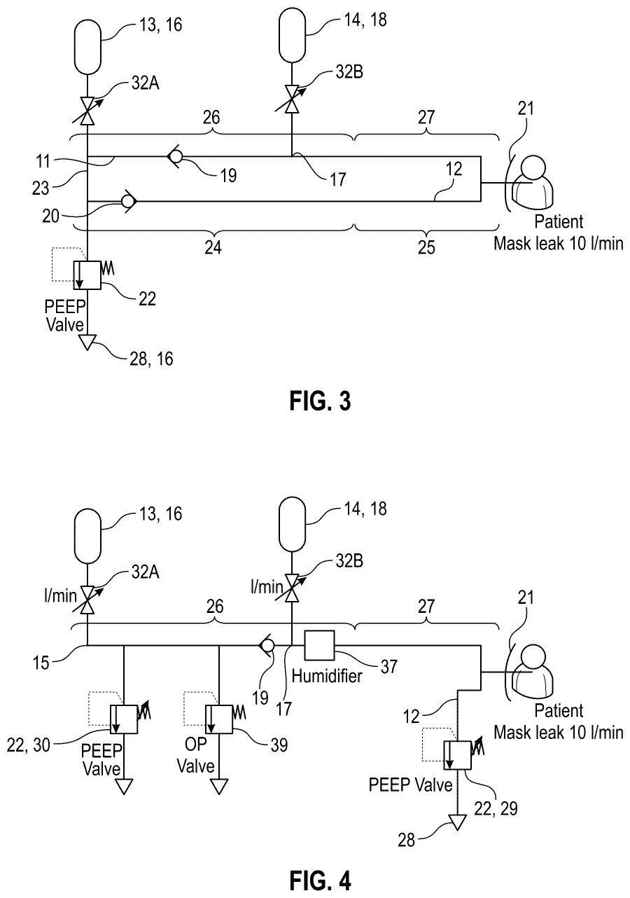

With reference to FIGS. 1, 3, 5, 6 and 10 a first gas source 13 is connected to a distal portion 26 of the inspiratory tube 11 at a first gas inlet 15 and a second gas source 14 is also connected to a distal portion 26 of the inspiratory tube 11 at second gas inlet 17. The first and second gas inlets 15 and 17 may include any suitable tube coupling, including an inline joiner, an L-shaped joiner, a T-shaped joiner and a Y-shaped joiner. The first and second gas sources 13 and 14 also include a proportional valve for controlling the flow rate of the first and second gases 16 and 18 entering the inspiratory tube 11. As can be seen, a first non-return valve 19 is located between the first and second gas inlets 15 and 17. That is to say, the first non-return valve 19 is located remotely from the proximal portion 27 of the inspiratory tube 11, or similarly, the first non-return valve 19 is located in the distal portion 26 of the inspiratory tube 11. Although not illustrated in FIG. 1, the first non-return valve 19 could also be located in an intermediate portion, that is between distal or proximal portions 26 and 27 of the inspiratory tube 11. In any event, the first gas 16 enters the inspiratory tube 11 upstream of the first non-return valve 19 and the second gas 18 enters the inspiratory tube 11 downstream of the first non-return valve 19. FIG. 3 comprises the same elements as FIG. 1, and like FIG. 1, the second gas inlet 17 and, indeed, the second gas 18 enters the inspiratory tube 11 in the distal portion 26.

The breathing circuit 10 includes a pressure regulation device 22 for regulating the pressure of the breathing gas within the breathing circuit 10. In the case of FIG. 1, the pressure regulation device 22 includes a first pressure relief valve 29 including a first positive end expiratory pressure valve (first PEEP valve) located on the expiratory tube 12 for venting exhaled gas(es). The first PEEP valve may be an adjustable valve that can be adjusted to open at pre-selected pressures. The first pressure relief valve 29 may be any suitable device including a fixed pressure relief valve, an orifice valve or a constant flow device that maintains a substantially constant flow irrespective of the pressure differential across the device 10. The expiratory tube 12 also includes a second non-return valve 20 upstream of the first PEEP valve to inhibit exhaled gas(es) from re-entering the patient interface and to inhibit the first gas from entering the expiratory tube 12. The pressure regulation device 22 may also include control valve 32B for controlling the delivery of the second gas 18 to the inspiratory tube 11, which may include the pressure of the second gas 18. Similarly, the pressure regulation device 22 may also include control valve 32A for controlling the delivery of the first gas 16 to the inspiratory tube 11, which may include the pressure of the first gas 16.

During patient inhalation and exhalation the first gas 16 is supplied to the inspiratory tube 11 upstream of the first non-return valve 19 and the second gas 18 is supplied at a constant flow rate to the inspiratory tube 11 downstream of the first non-return valve 19. That is to say, both the first gas 16 and the second gas 18 enter the distal portion 26 of the inspiratory tube 11.

At the start of patient exhalation, that is to say spontaneous exhalation, or during a pause between inhalation ending and exhalation starting, the first non-return valve 19 closes and the second gas 18 fills the gas passageway of the inspiratory tube 11 in a direction from the distal portion 26 toward the proximal portion 27, that is, in a direction toward the user. Although the first non-return valve 19 may be biased to a closed position, ideally the second gas 18 is supplied at a slightly higher pressure, for example in the range of about 1 to 300 cmH₂O greater than the pressure of the first gas 16, suitably about 1 to 250 cmH₂O greater, more suitably about 1 to 200 cmH₂O greater, more suitably about 1 to 150 cmH₂O greater, more suitably about 1 to 100 cmH₂O greater, more suitably about 1 to 50 cmH₂O greater, more suitably about 2 to 10 cmH₂O greater. While the first non-return valve 19 is closed, the first gas 16 is conveyed from the inspiratory tube 11 to the expiratory tube 12 via the bypass tube 23.

During patient exhalation, the second gas 18 flows along the inspiratory tube 11, displacing the first gas 16 and the second gas 18, or a mixture thereof, downstream of the second gas 18 entering the inspiratory tube 11, and thereby storing a volume of the second gas 18 in the inspiratory tube 11 during patient exhalation. The first gas 16 and the second gas 18 displaced from the inspiratory tube can be conveyed from the inspiratory tube 11 into the expiratory tube 12 with exhaled gas(es) 28. Exhaled gas(es) 28 flow from the patient interface 21 into the expiratory tube 12 and away from the patient interface 21. Once the exhaled gas(es) 28 pass the second non-return valve 20 they can be vented from the breathing circuit 10. Similarly, excess supply of the first gas 16 flowing from the inspiratory tube 11 to the expiratory tube 12 can also be vented.

During patient inhalation, that is to say spontaneous inhalation, the second non-return valve 20 closes and the first non-return valve 19 opens. In a situation in which the internal volume of the inspiratory tube 11 between the first non-return valve 19 and the patient interface 21 approximates the tidal volume of the user, during inhalation the user initially receives a dose of the second gas 18 which is drawn into the alveoli of the user's lungs. In the event that the internal volume of the inspiratory tube 11 is less than the tidal volume of the user, or the flow rate of the second gas 18 into the inspiratory tube 11 is insufficient to fill the inspiration tube 11 with a tidal volume of the user, after the initial inhalation phase, the user may receive a combination of the first and second gases 16 and 18. This may not be disadvantageous as the first and second gases 16 and 18 may not go beyond the upper respiratory passage of the user where no gas transfer occurs. Conversely in the event that the internal volume of the inspiratory tube 11 is larger than the tidal volume of the user, and indeed, a volume of the second gas 18 loaded into the inspiratory tube 11 during the exhalation phase is greater than the tidal volume of the user, not all of the second gas 18 that is loaded/stored in the inspiratory tube 11 during exhalation will be inhaled. At the start of inhalation by the user, the first non-return valve 19 opens, allowing both the first gas and the second gas 16 and 18 to be supplied into the distal portion 26 of the inspiratory tube 11 which flows in a direction toward the user.

One of the advantages of this embodiment is that the second gas 18 fills the inspiratory tube 11 from the distal portion 26 and any patient interface leak that occurs during exhalation comprises the first gas 16 and a small amount of the second gas 18 that filled the inspiratory tube 11 at the end of the previous inhalation. This minimises any patient interface leak of the second gas 18 from the breathing circuit 10 and allows a lower flow rate of the second gas 18 to achieve an effective therapy.

FIG. 2 is an example of a breathing circuit 10 in which the proximal portions 27 and 25 of the inspiratory and expiratory tubes 11 and 12 respectively are interconnected and the distal portions 26 and 24 of the tubes 11 and 12 are not connected. However, like the examples shown in FIGS. 1 and 3, the second gas 18 enters the distal portion 26 of the inspiratory tube 11 downstream of the first non-return valve 19. During patient exhalation the first non-return valve 19 closes and the second gas 18 flows along the inspiratory tube 11, displacing the first gas 16 and the second gas 18, or a mixture thereof downstream of the second gas 18 entering the inspiratory tube 11 into the expiratory tube 12, thereby loading a volume of the second gas 18 in the inspiratory tube 11. Although not shown in FIG. 2, the expiratory tube 12 may be omitted and a pressure regulation device 22 such as a PEEP valve could be fitted to an outlet port of the patient interface 21 for venting exhaled gas 28.

During patient inhalation, that is to say spontaneous inhalation, the first non-return valve 19 opens. The user initially receives a dose of the second gas 18 that was loaded into the inspiratory tube 11 during exhalation and is drawn into the alveoli of the user's lungs. In addition, at the start of inhalation by the user, the first non-return valve 19 opens, allowing both the first gas 16 and the second gas 18 to be supplied into the distal portion 26 of the inspiratory tube 11 which flows in a direction toward the user, and in the event that the volume of the second gas 18 loaded into the inspiratory tube 11 is less than the tidal volume, a mixture of the first and second gases 16 and 18 can be supplied and received by the user. The first and second gases 16 and 18 supplied to the user toward the end of the inhalation cycle is usually received within the upper regions of the inspiratory passage where gas transfer does not occur.

Although not shown in the Figures, the breathing circuit 10 may also include a sensor to detect the amount of oxygen gas being inhaled by the patient. An example of a suitable sensor is a galvanic oxygen sensor to determine the fraction of inspired oxygen gas (FiO₂) over 15 seconds or more, as this would effectively filter the FiO₂ to a stable, indicative value of the FiO₂ over the entire breath. Adjustments can then be made to the flow rates of the first and second gases 16 and 18 to the inspiratory tube 11. Alternatively, an ultrasonic sensor can be used to take rapid readings for detecting FiO₂ during the course of each breath. The sensor may be located at any suitable location in the breathing circuit. Suitable locations include the patient interface or immediately upstream of the patient interface.

The pressure regulation device 22 of the breathing circuit 10 shown in FIG. 2 also includes a second pressure relief valve 30 in the distal portion 26 of the inspiratory tube 11. The second pressure relief valve 30 includes a second positive end expiratory pressure valve (second PEEP valve) upstream of the first non-return valve 19 which vents the first gas 16. The first gas 16 can be supplied at any rate and is expected to be supplied at a rate equal to, or greater than the peak inspiratory flow rate.

In addition, the pressure regulation device 22 of the breathing circuit 10 shown in FIG. 2 also includes a third pressure relief valve including an overpressure valve 39 for venting breathing gases from the inspiratory tube 11 in the event of a blockage or malfunction. The overpressure valve 39 has a setting that is greater than the operating pressure of the first pressure relief valve 29 and, if present, the second pressure relief valve 30. In any event, the overpressure valve 39 is located upstream of the first non-return valve 19 and downstream of the second pressure relief valve 30.

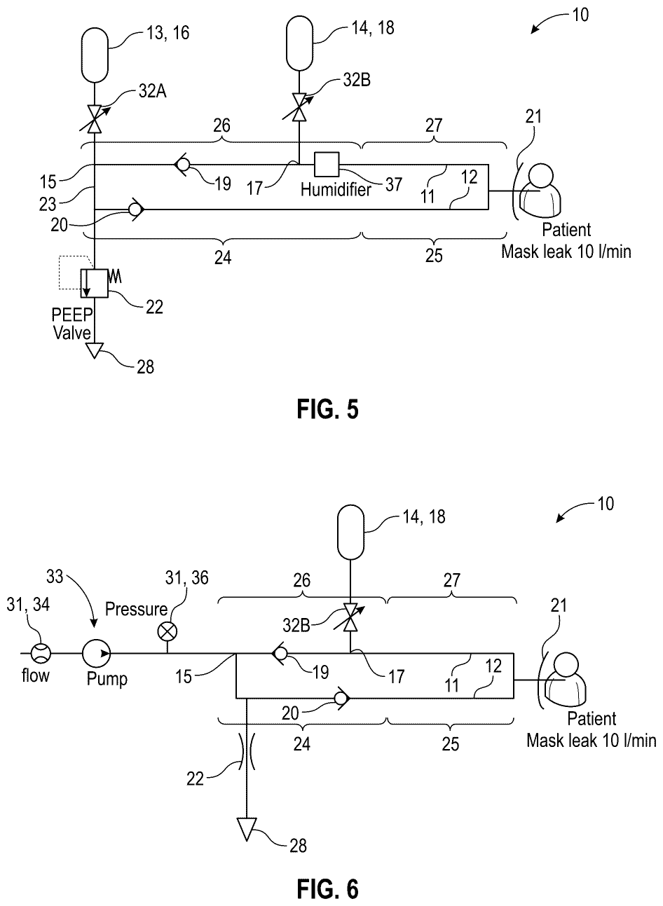

FIGS. 4 and 5 are the same as the breathing circuits 10 shown in FIGS. 2 and 3 respectively and only differ with the inclusion of a humidification device 37 located downstream of the second gas inlet 17. The humidification device 37 may be any suitable device in which the breathing gas contacts a path of heated water for humidifying the breathing gas. In any event, the internal volume of the humidification device 37 will also form part of the internal volume of the inspiratory tube 11, meaning that the second gas 18 will be loaded into and accumulate in the humidification device 10 during exhalation by the user. That is to say, the internal volume of the humidification device 37 occupied by gas may be taken into account when determining the internal volume of the inspiratory tube 11.

One of the advantages in locating the humidification device 37 downstream of the second gas inlet 17 is that both the first and second gases 16 and 18 are humidified by the humidification device 37. One of the benefits of this configuration is that the first non-return valve 19 is located upstream of the humidification device 37 meaning that the first non-return valve 19 will remain dry and therefore the humidification device 37 will not potentially affect the reliability of the first non-return valve 19. In addition, any secretions from the user are unlikely to pass through the humidification device 37 and reach the first non-return valve 19. In other words, the humidification device 37 provides a further obstacle to secretions from the user reducing the reliability of the breathing circuit 10.

If desired, the first and second gases 16 and 18 could be humidified in separate humidification devices. For example, although not shown in the drawings, a humidification device could be located in the inspiratory member 11 between the first and second gas inlets 15 and 17 for humidifying the first gas 16. A separate humidification device generator 33. The pressure sensor 36 could also be located at or close to the patient interface 21 and/or anywhere in the inspiratory tube 11 downstream of the flow generator 33, it may also be downstream of a blower within the flow generator.

In addition, a flow sensor 34 may be located at or close to the inlet of the flow generator 33, which enables the flow generator 33 to be arranged as a single self-contained module. Although not illustrated, the flow sensor 34 could also be located at the outlet of the flow generator 33. Moreover, the flow sensor 34 can be located anywhere in the inspiratory tube 11, expiratory tube 12, or upstream or downstream of a blower within the flow generator 33. In any event, outputs from the sensors 34, 36 can be used to operate the flow generator 33, including increasing or decreasing the speed of the flow generator 33 as desired. However, in the case of the respiratory therapy, it is desirable to control the pressure at the patient interface. In one example, the variable flow regulator 33 can be used to provide an inspiratory pressure, such as an inspiratory positive airway pressure (IPAP) and an expiratory pressure, such as an expiratory positive airway pressure (EPAP), or continuous positive airway pressure (CPAP), in which IPAP equals EPAP. The inspiratory pressure and expiratory pressure can be preselected using an operating interface on the variable flow generator 33 and the pressure and flow sensors 36, 34 located at the flow generator 33 can be used to determine when the patient begins inhalation and exhalation.

A controller can also be used to estimate pressure drop within sections of the breathing circuit 10, which may be a function of the flow rate, to estimate the pressure at the patient interface based on the signal outputs of the flow sensor 34 and the pressure sensor 36 of the variable flow generator 33. For instance, the controller can estimate the pressure drop in the inspiratory tube 11 based on pre-determined components for specific breathing circuits 10. That is to say, different pre-determined functions, such as humidification devices 37, non-return valves 19, length of the inspiratory tube 11, internal diameter of the inspiratory tube 11 and so forth. In any event, the pressure drop will be a function of the various components and layout of the breathing circuit 10 and the components can be selected as desired.

The variable flow generator 33 enables the pressure requirements for effective therapy to be delivered whilst reducing or minimizing the excess supply of the first gas to specific operating ranges. This means that pressure regulation of the breathing circuit 10 can be achieved using devices that are less pressure dependent or are not pressure dependent at all. For instance, one of the advantages in using a variable flow generator 33 as a first gas source 13 is that the pressure regulation device 22 can be modified by replacing pressure relief valves, such as a PEEP valve with a simpler structure such as a flow restrictor, an orifice of fixed area or a constant flow valve. A constant flow valve may have a variable orifice that changes with the application of pressure so that flow rate through the valve remains substantially constant despite change in pressure across the orifice.

In another example, the flow sensor 34 may be located on the inspiratory tube 11 to detect the flow being inhaled by the patient. An output of the flow sensor 34 may then be used to determine when inhalation starts and ends which can be used to operate the variable flow generator 33 at either the inspiratory pressure or the expiratory pressure.

In another example, the flow sensor 34 may be located on the expiratory tube 12 to detect the flow being exhaled by the patient. An output of the flow sensor 34 may then be used to determine when exhalation starts and ends which can be used to operate the variable flow sensor 34 at either the inspiratory pressure or the expiratory pressure.

Advantages in using a variable flow generator 33 and a simplified pressure regulation device 22 include the following:
i) Minimal flow rates over the pressure ranges can be achieved.
ii) Minimal flow rates minimises the amount of the first gas 16 exiting the breathing circuit 10 and thus the total flow through the circuit 10.
iii) This has associated benefits of reducing power usage, reduces wear and deterioration on the components of the circuit 10, reduces noise and decreases the humidification load in the event that the breathing circuit includes a humidification device 37.
iv) In addition, battery-powered flow generators 33 with small throughputs can be used. This enables the circuits 10 to be used in ambulances and by other first responders without using mains power.

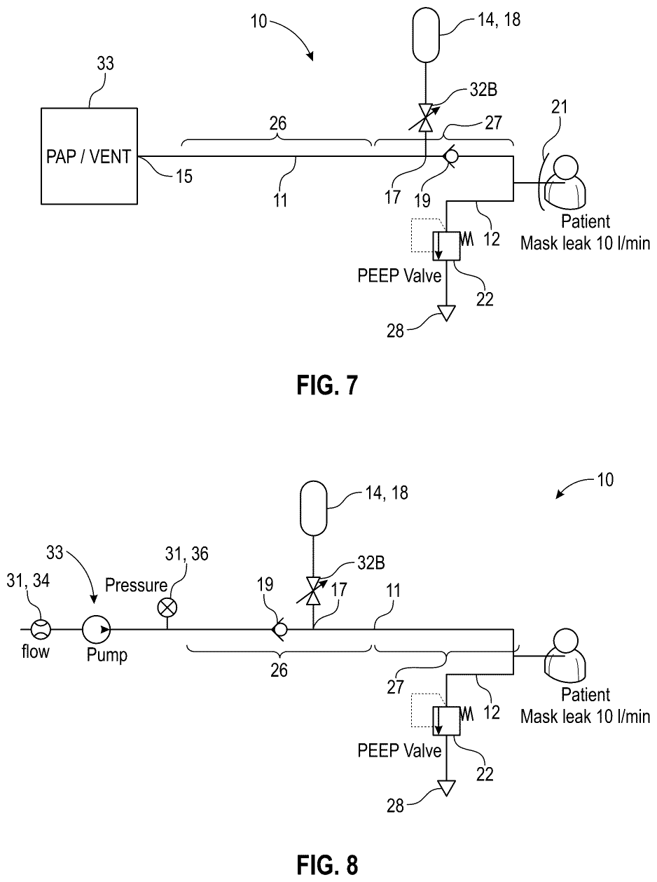

FIG. 7 is an example of a breathing circuit 10 in which the first gas source 13 includes a variable flow generator 33 connected to a distal portion 26 of the inspiratory tube 11, a first gas inlet 15 located at a distal portion 26 of the inspiratory tube 11, and a first non-return valve 19 located downstream of the first gas inlet 15 and downstream of a second gas inlet 17. The flow generator 33 can be set to deliver a set pressure, including an inspiratory pressure and an expiratory pressure. The inspiratory pressure may be higher than the expiratory pressure. The flow generator 33 can have sensor 31 at various locations as described in relation to FIG. 6. An advantage in this configuration is that the flow rate of the first gas 16 supplied can be controlled to increase when the user inhales and reduce when the user exhales. As a result the total flow of the first gas 16 can be more closely matched to the flow and pressure required to provide the positive pressure respiratory therapy required. Moreover, the pressure regulation device 22 of the breathing circuit 10 can be simplified to a first pressure relief valve 29 of the expiratory tube 12, removing the need for the second pressure relief valve of the inspiratory tube 11. The first pressure relief valve 29 should be set at pressure slightly above the maximum pressure of the variable flow generator 33, to minimize the second gas 18 being vented from the expiratory tube 12 without being inhaled by the user.

During patient inhalation and exhalation, the second gas 18 enters the proximal portion 27 of the inspiratory tube 11 via the second gas inlet 17 at a constant rate, and the first gas 16 enters the distal portion 26 via the first gas inlet 15. Control valve 32B can be used to control the flow and/or pressure of the second gas 18 at the second gas inlet 17. At the start of patient exhalation, or during a pause between inhalation ending and exhalation starting, the first non-return valve 19 closes and the second gas back fills the gas passageway of the inspiratory tube 11 in a direction from the proximal portion 27 toward the distal portion 26. During patient exhalation, the second gas 18 and the first gas 16 forms a gas/gas interface that moves along the gas passageway away from the second gas inlet 17 toward the distal portion 26, thereby storing a volume of the second gas 18 in the inspiratory tube 11 during patient exhalation. An amount of the first gas 16 may be vented from the inspiratory tube 11 during this stage by, for example, the variable flow generator 33. The volume of the second gas 18, such as oxygen, that enters the inspiratory tube 11 during exhalation may be equal to, or less than, a tidal volume of the user, thereby minimizing wastage of the second gas 18 by avoiding venting the first gas 16 during exhalation.

During patient inhalation, the first non-return valve 19 opens, and the second gas 18 that has been stored in the inspiratory tube 11 flows into the patient interface 21. If all of the second gas 18 is inhaled, the user will begin to inhale a mixture of the first gas 16 and the second gas 18. The total volume of the second gas 18 loaded and stored in the inspiratory tube 11 may be adjusted and controlled based on the internal volume of the inspiratory tube 11 and the flow rate of the second gas 18.

FIG. 8 is the same as the breathing circuit 10 shown in FIG. 7 with the exception of the second gas inlet 17 being located in a distal portion 26 of the inspiratory tube 11 and downstream of the first non-return valve 19. During inhalation, the second gas 18 flows along the inspiratory tube 11 toward the user in the same manner described in relation to the breathing circuit 10 of FIGS. 1 to 6.

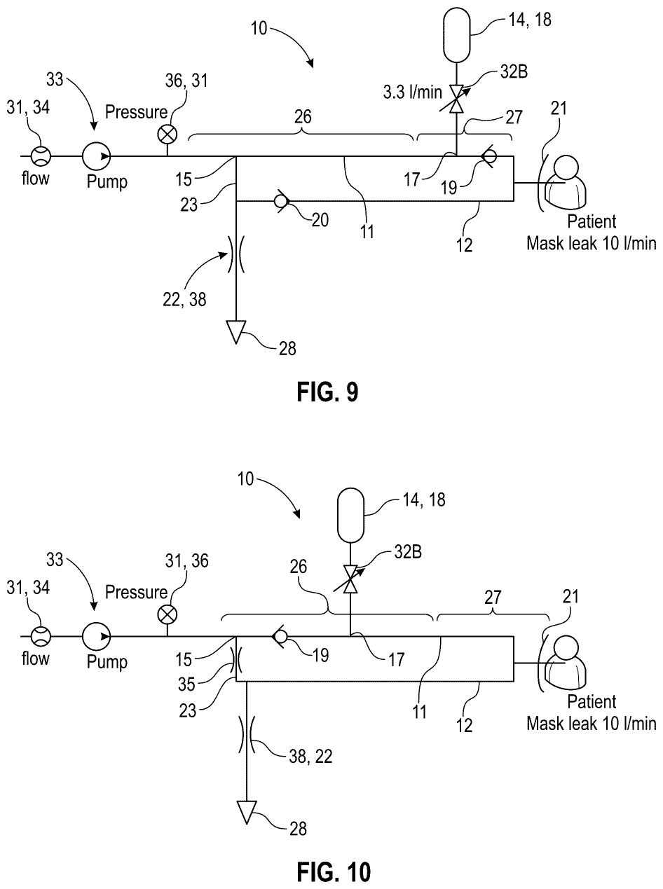

FIG. 9 is an example of breathing circuit 10 in which the distal portions 26 and 24 of the inspiratory and the expiratory tubes 11 and 12 are connected, and the proximal portions 27 and 25 of the tubes 11 and 12 are connected. The breathing circuit 10 also has a variable flow generator 33, a second gas inlet 17 in the proximal portion 27 of the inspiratory tube 11, a first non-return valve 19 downstream of where the second gas 18 enters the inspiratory tube 11, and a second non-return valve 20 in the expiratory tube 12 that inhibits the first gas 16 from entering patient interface from the expiratory tube 12. The variable flow generator 33 can be operated and have sensors 31 located on the breathing circuit as described in relation to FIG. 6. A control valve 32B may control the flow and/or pressure of the second gas 18 at the second gas inlet 17. As can be seen, a bypass tube 23 interconnects the distal portions 26 and 24 of the inspiratory and expiratory tubes 11 and 12 for conveying any excess supply of the first gas 16 to the expiratory tube 12.

The variable flow generator 33 can be operated so that pressure output of the flow generator 33 more closely corresponds to the inspiratory pressure requirement and the expiratory pressure requirement of the user. The breathing circuit has a pressure regulation device 22 including a flow restrictor 38 in a distal portion of the expiratory tube 12 for venting exhaled gas 28 and any excess supply of the first gas. The flow restrictor 38 may include an orifice that can regulate venting of the first gas 16 and the exhaled gas from the expiratory tube 12. In the event that the first and second gases 16 and 18 are supplied at the required amounts, the excess supply of the first gas 16 will be minimized and little or no excess first gas will be conveyed by the bypass tube 23 and vented.

The flow of the first and second gases 16 and 18 in the inspiratory tube 11 in FIG. 9 is much the same as that described in relation to FIG. 7. Specifically, during patient inhalation and exhalation, the second gas 18 enters the proximal portion 27 of the inspiratory tube 11 via the second gas inlet 17 at a constant rate, and the first gas 16 enters the distal portion 26. At the start of patient exhalation, or during a pause between inhalation ending and exhalation starting, the first non-return valve 19 closes. Once closed, and throughout exhalation a volume of the second gas 18 back fills the inspiratory tube 11 in a direction from the proximal portion 27 toward the distal portion 26. To accommodate the second gas 18 back filling the inspiratory tube 11, the first gas 16 upstream of the second gas 18 in the inspiratory tube 11 can flow to the expiratory tube 12 via the bypass tube 23. In addition, the second non-return valve 20 opens and exhaled gas is conveyed through the second non-return valve 20 and vented from the circuit 10.

During inhalation, the first non-return valve 19 opens and the user initially inhales the volume of the second gas 18 loaded and stored in the inspiratory tube 11 during exhalation. Once the volume of the second gas 18 has been inhaled, and if the patient continues to inhale the user receives a mixture of the second gas 18 and the first gas 16 that simultaneously enters the inspiratory tube 11.

One of the elements of the breathing circuit 10 shown in FIG. 9 is that the second non-return valve 20 inhibits the first gas 16 from entering the patient interface from the expiratory tube 12. However, by controlling the output pressure of the flow generator 33 during inhalation, the flow generator 33 can output a respiratory pressure that corresponds to the inspiratory therapy pressure required by the user, in which case the second non-return valve 20 may not be needed. FIG. 10 is an example of a breathing circuit 10 where the second non-return valve 20 has been omitted. However, to inhibit the first gas 16 from being conveyed through the bypass tube 23 to the patient interface via the expiratory tube 12 during patient inhalation, an additional flow restrictor device 35 is provided in the bypass tube 23 to inhibit the first gas 16 from freely flowing to the patient interface via the bypass tube 23 and the expiratory tube 12. The additional flow restrictor device 35 also allows the first gas to flow to the expiratory tube 12 and be vented during patient exhalation. The flow restrictor device 38 may be any device having a reduced flow cross-section compared to the inspiratory tube 11.

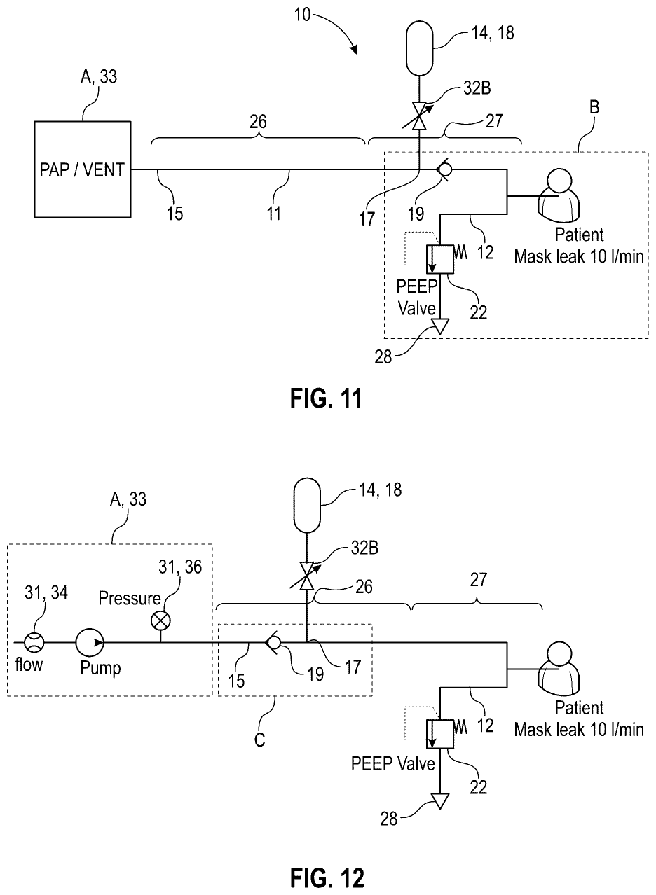

FIGS. 11 to 15 are examples of breathing circuits 10 in which at least some of the elements are arranged in one or more modules to reduce manual assembly by a clinician and increase the ease of use of the circuit 10. The elements may be integrated in a preassembled operative configuration, or partially preassembled to assist in setting up of the circuit 10. The elements that are not assembled may have markings indicating which elements to interconnect, for example, the first gas inlet 15 may be a linear joiner that includes markings to indicate which port to connect to the first gas source 13 and another port to connect to a distal portion 26 of the inspiratory tube 11. In another example, the second gas inlet 17 may be a T-shaped joiner having a first limb marked for connection to the second gas source 14, a second limb marked for connection to distal or proximal portions 26 and 27 of the inspiratory tube 11, and a third limb for connection to a first non-return valve 19. In FIGS. 11 to 17, the modules are represented by the boxes shown in dotted lines, except for the module in FIG. 11 which includes a second module shown in the solid lines. A summary of the modules is as follows.

Module A shown in FIG. 11 is a positive air pressure device (PAP device) or ventilator, identified by the solid box. The PAP device or ventilator, including non-invasive ventilators may include an integrated flow generator 33, a flow sensor 34 and a pressure sensor 36. The flow generator 33, may comprise a blower and may have sensors 31 as described in relation to FIG. 6. The flow generator 33 may be operated over pressure settings between an inspiratory pressure (IPAP) and an expiratory pressure (EPAP) that can be selected for particular users, or the flow generator 33 can be operated at a constant pressure setting. The constant pressure setting can be reset as and when required. Module B in FIG. 11 includes a proximal portion 27 of the inspiratory tube 11, a second gas inlet 17 including a first three limb joiner, in which a first limb of the joiner is connectable to a second gas source 14, a second limb is connectable to a distal portion 26 of the inspiratory tube 11, and a third limb of the joiner is connectable to a first non-return valve 19. A three-limb joiner having a first limb connectable to the outlet of the first-non-return valve 19 or a tube extending from the first non-return valve 19, a second limb connectable to a distal portion 24 of the expiratory tube 12, and a third limb connected to the patient interface. A pressure relief valve is connected to the expiratory tube 12. Although FIG. 11 illustrates the patient interface 21 within the dotted lines representing Module B, the patient interface 21 may be disconnected from the module initially, allowing a patient interface 21 suitable for the user to be selected.

FIG. 12 illustrates Module A including a PAP device and Module C that is connectable to Module A. The connection between Modules A and C may be provided by a linear tube joiner. Module C includes a T-shaped joiner having one limb connectable to a second gas source 14, a second limb connectable to the outlet of the first non-return valve 19, and a third limb connectable to a length of the inspiratory tube 11 downstream of Module C.

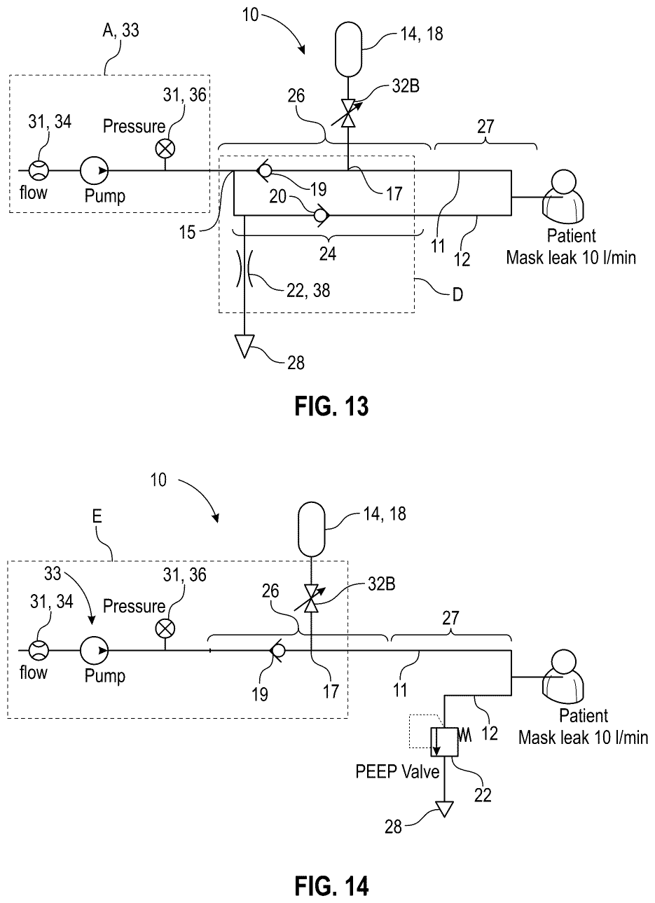

FIG. 13 illustrates Module A including the PAP device and Module D which includes distal portions of the inspiratory and expiratory tubes 11 and 12 interconnected a bypass tube 23 for conveying the first gas 16 supplied in excess to the inspiration tube 11 to the expiratory tube 12. In respect of Module D the distal portion 26 of the inspiratory tube 11 includes a non-return valve 19 inhibiting flow in a direction away from the user and a T-shaped joiner which provides the second gas inlet 17. The T-shaped joiner includes first and second limbs connected to the distal portion of the inspiratory tube 11 and a third limb connectable to the second gas source 14. The expiratory tube 12 includes a pressure regulation device 22 suitably in the form of a restriction orifice, for venting exhaled gas and the first gas 16 supplied in excess to the circuit 10. The expiratory tube 12 also includes a second non-return valve 20 inhibiting the flow of gas in a direction toward the user. The distal portions 26 and 24 of the inspiratory tube 11 and the expiratory tube 12 within Module D can be connected to the inspiratory tube 11 and the expiratory tube 12 outside of Module D using inline joiners.

FIG. 14 illustrates Module E including a PAP device and a distal portion 26 of the inspiratory tube extending from the outlet of the PAP device. The PAP device includes a flow generator 33 such as a blower, a pressure sensor 36 downstream of the blower, for example, at or near the outlet of the blower. The pressure sensor 36 could also be located at or close to the patient interface 21 and/or anywhere in the inspiratory tube 12 downstream of the flow generator 33. The pressure sensor 36 may also be downstream of a blower within the flow generator. A flow sensor 34 may be located at or near the inlet of the blower. Moreover, the flow sensor 34 can be located anywhere in the inspiratory tube 11, expiratory tube 12, or upstream or downstream of a blower within the flow generator 33. The blower being operable at a constant positive pressure or at a variable positive pressure to suit the breathing cycles of the user, including a higher pressure during patent inhalation and a lower pressure during exhalation. The distal portion 26 of the inspiratory tube 11 includes a first non-return valve 19 that inhibits flow in a direction toward the outlet of the blower and a T-joiner downstream of the first non-return valve 19, which provides the second gas inlet 17. One limb of the T-joiner is connected downstream of the first non-return valve 19, another limb being connected to a proportional control valve for controlling the pressure and flow rate of the second gas 18 supplied by second gas source 14 and a third limb which may be connected to a section of the distal portion 26 of the inspiratory tube 11, or alternatively, the third limb may be connected to the remainder of the inspiratory tube 11 extending to the user.

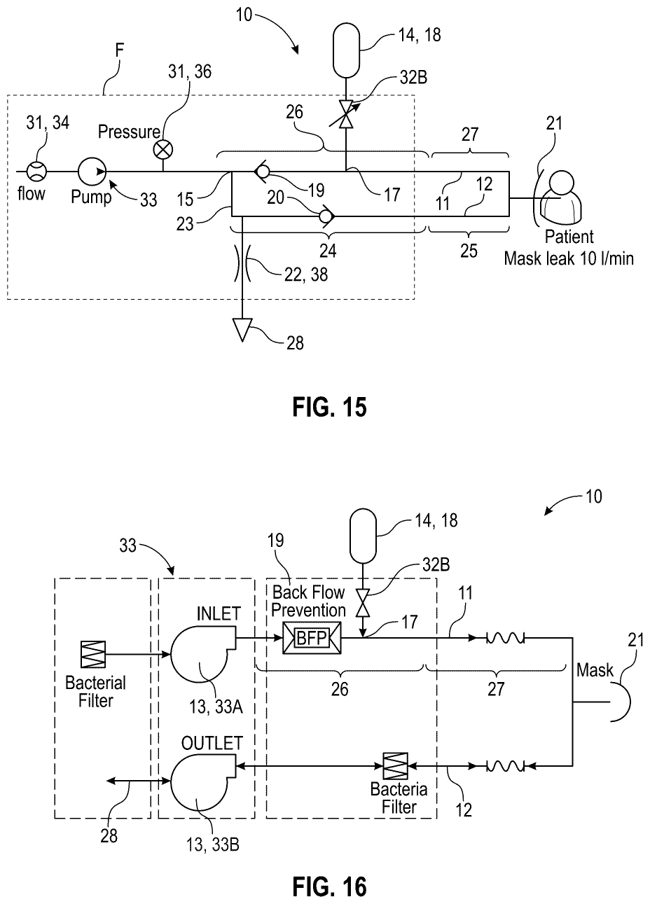

FIG. 15 illustrates Module F including a PAP device and distal portions 26 and 24 of the inspiratory and expiratory tubes 11 and 12 interconnected by a bypass tube 23 that conveys the first gas 16, supplied in excess to the breathing circuit 10, to the expiratory tube 12. The PAP device includes a flow generator 33, which may have a blower. The flow generator 33 includes sensor 36 downstream of the blower, for example at or near the outlet of the blower. Moreover the pressure sensor 36 could also be located at or close to the patient interface 21 and/or anywhere in the inspiratory tube 12 downstream of the flow generator 33. The pressure sensor 36 may also be downstream of a blower within the flow generator 33. A flow sensor 34 may be located at or near the inlet of the blower. Moreover, the flow sensor 34 can be located anywhere in the inspiratory tube 11, expiratory tube 12, or upstream or downstream of a blower within the flow generator 33. The blower being operable at a constant positive pressure or at a variable positive pressure to suit the breathing cycles of the user, including a higher pressure during patent inhalation and a lower pressure during exhalation. The distal portion 26 of the inspiratory tube 11 includes a first non-return valve 19 that inhibits flow in a direction toward the outlet of the blower and a T-joiner downstream of the first non-return valve 19. One limb of the T-joiner is connected downstream of the first non-return valve 19, another limb being connected to a proportional control valve for controlling the pressure and flow rate of the second gas 18 supplied by second gas source 14 and a third limb which may be connected to a section of the distal portion 26 of the inspiratory tube 11, or alternatively, the third limb may be connected to the remainder of the inspiration tube extending to the user. The expiratory tube 12 includes a restrictor suitably in the form of a restriction orifice, for venting exhaled gas and the first gas supplied in excess to the circuit. The expiratory tube 12 also includes a second non-return valve 20 inhibiting the flow of gas in a direction toward the user. Ends of distal portions 26 and 24 of the inspiratory tube 11 and the expiratory tube 12 of Module F can be connected to the inspiration tube 11 and the expiratory tube 12 outside of Module F using inline joiners.

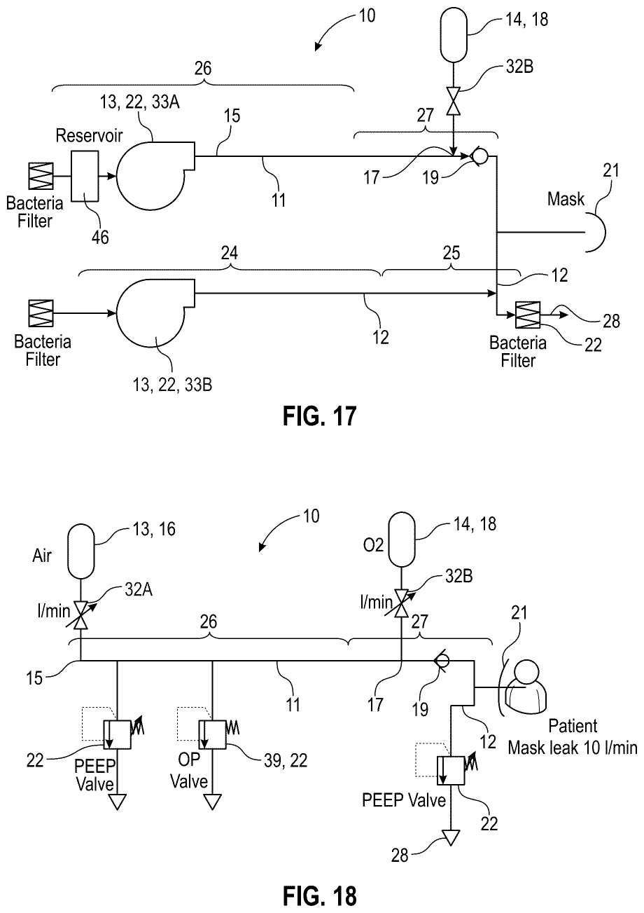

FIGS. 16 and 17 illustrates a breathing circuit 10 including a flow generator 33 having first and second flow generators 33A and 33B respectively connected to distal portions 26 and 24 of the inspiratory and expiratory tubes 11 and 12 respectively. The first and second flow generators 33A and 33B of FIGS. 16 and 17 can be operated in a similar manner.

As shown by one of the dashed outlines in FIG. 16, the first and second flow generators 33A and 33B can be integrated into a single module that may be contained within a single housing. The first flow generator 33A and second flow generator 33B can be operated in combination to provide a constant positive flow or a variable positive flow to conform to the inspiratory pressure (IPAP) and the expiratory pressure (EPAP) of the patient. For instance, the first flow generator 33A can be operated as a first gas source 13, and the flow generator 33B can be operated to provide back pressure to the expiratory tube 12 during patient exhalation.

Although not illustrated in FIG. 16, the breathing circuit 10 may include single or multiple pressure sensors and, optionally, a single or multiple flow sensors. The pressure sensors may be located on the patient interfaces 21 or downstream of the flow generators 33A and 33B. Moreover the pressure sensor could also be located at or close to the patient interface 21 and/or anywhere in the inspiratory tube 12 downstream of one or both of the flow generators 33A and 33B. The pressure sensor may also be downstream of a blower of one or both of the flow generators 33A and 33B. In addition, flow sensors (not shown) could be located either upstream or downstream of the flow generators 33A and 33B. Moreover, the flow sensor(s) 34 can be located anywhere in the inspiratory tube 11, expiratory tube 12, or upstream or downstream of a blower within one or both of the flow generators 33A and 33B. Outputs from the sensor(s) can be used to operate the flow generator 33A and 33B such that constant or variable pressure is provided by controlling the flow generator 33B, while the first flow generator 33A is controlled to provide flow during the inspiratory phase of the breath. The inlet of the first flow generator 33A may include a bacterial filter, and a bacterial filter may also be located in the expiratory tube 12. The inspiratory tube 11 downstream of the outlet of the first flow generator 33A also includes a first non-return valve 19 inhibiting flow from the second flow generator 33B toward the outlet of the first flow generator 33A. The inspiratory tube 11 also includes a port that is connectable to a second gas source 14 that may be positioned upstream or downstream of the non-return valve 19. The inspiratory and expiratory tubes 11 and 12 may have lengths that can be selected by the length adjuster to provide the required internal volume of the inspiratory tube 11 and enable components of the breathing circuit 10, such as the second gas source 14, control valves 32B, non-return valve 19 and bacteria filters and so forth to be positioned at a distance away from the user so as to prevent weight of the elements from pulling on the patient interface 21. In addition to the option of the first and second flow generators 33A and 33B being arranged in a single housing, the non-return valve 19 and the bacterial filters may also be located within the same housing or module as the first and second flow generators 33A and 33B. Similarly, if desired the port for connection to the second gas source may also be included in the same housing or module.

FIG. 17 illustrates a breathing circuit 10 including a flow generator 33 having first and second flow generators 33A and 33B respectively connected to distal portions 26 and 24 of the inspiratory and expiratory tubes 11 and 12 respectively. The first and second flow generators 33A and 33B can be integrated into a single module that may be contained within a single housing. The first flow generator 33A provides the first gas source 13, and the second gas source 14 is connected to proximal portion 27 of the inspiratory tube 11 at a second gas inlet 17. A first non-return valve 19 is located downstream of the second gas inlet 17. A reservoir for storing the second gas 18 is located upstream of the first flow generator 33A, however, the reservoir could also be located downstream of the first flow generator 33A. A pressure regulation device 22, including a restricted orifice is provided in the expiratory tube 12 for venting exhaled gas 28. The pressure regulation device may have a bacterial filter. Bacterial filters may also be provided at or near inlets to the first and second flow generators 33A and 33B. The first flow generator 33A and second flow generator 33B can be operated in combination to provide a constant positive flow or a variable positive flow to conform to the inspiratory pressure (IPAP) and the expiratory pressure (EPAP) of the patient.

Specifically, during patient exhalation the second flow generator 33B is operated to provide a back pressure, for example 10 cmH₂0, that provides EPAP for the patient. The first flow generator 33A is operated at a lower pressure set point, or at a lower controlled flow, such that the first non-return valve 19 in the inspiratory tube 11 remains closed. This allows the second gas 18 to enter via second gas inlet 17 and accumulate and be stored in the inspiratory tube 11 by flowing in a direction away from the patient interface 21. The second gas 18 may also be stored in the reservoir, which as mentioned may optionally be placed upstream of the first flow generator 33A. Exhaled gas(es) 28 are vented from the expiratory tube 12 via pressure regulation device 22 located in the proximal portion of the expiratory tube 12.

If there is any mask leak during expiration, the gas leaking is made up of either exhaled gas(es), and/or gas delivered by the second flow generator 33B. This means that no oxygen will leak during expiration, as the first non-return valve remains closed due to the lower pressure or flow provided by the first flow generator 33A. Exhaled gas that are not leaked from the patient interface vent from the circuit 10 to atmosphere proximal to the patient via a pressure regulation device 22, such as restricted orifice. The pressure regulation device may also include a bacterial filter as illustrated. An advantage this provides is that no carbon dioxide builds up in the expiratory tube. That is to say the expiratory tube 12 can be flushed during patient exhalation.

During patient inspiration, the first non-return valve opens, and the first gas, suitably air enters the distal portion of the inspiratory tube which displaces the second gas, suitably oxygen gas, to deliver the second gas to the patient, at the pressure (IPAP) set by the first flow generator 33A. During patient inhalation, the first flow generator 33A may have an increased or increasing pressure set point or controlled flow and the second flow generator 33B may have a reduced or decreasing pressure set point or controlled flow, so that the first flow generator 33A is at a higher pressure or flow setting than the second flow generator 33B. This ensures that the patient breathes in the first and second gases delivered from the inspiratory tube and not any gases from the second flow generator 33B.

Although not shown in FIG. 17, pressure and flow sensors may be arranged in the inspiratory tube 11, and outputs of these sensors can be used to operate the first flow generator 33A. Similarly, pressure and flow sensors may be arranged in the inspiratory tube 12, and outputs of these sensors can be used to operate the second flow generator 33B of the expiratory tube 12. The pressure sensors for either flow generator may also be located at the patient interface 21.

FIG. 18 is an example of a breathing circuit 10 in which the proximal portions 27 and 25 of the inspiratory and expiratory tubes 11 and 12 are interconnected. As can be seen, the second gas 18 enters the inspiratory tube 11 upstream of the first non-return valve 19 at a second gas inlet 17. Suitably the second gas inlet 17 and the first non-return valve 19 are located in a proximal portion 27 of the inspiratory tube 11. In comparison, the second gas 18 enters downstream of the first non-return valve 19 in FIGS. 1 to 6 and 8.

The first non-return valve 19 may be any suitable valve, including a one-way flap valve, a biased valve that is biased into a closed position, or a diaphragm valve. The first non-return valve 19 closes when the gas pressure downstream of the first non-return valve 19, for instance in the patient interface during exhalation, is greater than the pressure upstream in the inspiratory tube 11. The first non-return valve 19 opens, suitably automatically, when the patient spontaneously inhales.

The breathing circuit 10 includes a pressure regulation device 22 having first and second pressure relief valve located in the expiratory tube 11 and in the distal portion 26 of the inspiratory tube 11 respectively. The first pressure relief valve is a first positive end expiratory pressure valve (first PEEP valve) and the second pressure relief valve is a second positive end expiratory pressure valve (second PEEP valve). The second PEEP valve has setting marginally above the setting of the first pressure relief valve, for example, 0.5 to 2.0 cmH₂O greater.

The second PEEP valve and the overpressure relief valve 39 may be connected to the inspiratory tube 11 using any suitable tube joiner including a T-shaped joiner and Y-shaped joiner.

Proximal portions 25 and 27 of the expiratory tube 12 and the inspiratory tube 11 are connected to the patient interface 21 by a tube joiner having three limbs, such as a T-piece or a Y-piece, in which one of the limbs connects to the expiratory tube 12, another limb connects to the inspiratory tube 11, and a third limb of the tube connector couples to an inlet/outlet port on the patient interface 21 to conduct breathing gas as it is inhaled and exhaled. The tube joiner may be integrally formed with the patient interface 21. In another example, not illustrated in the Figures, the inspiratory tube 11 and the expiratory tube 12 may be directly connected to an inlet connection and outlet connection on the patient interface 21 respectively.

During patient inhalation and exhalation, the first gas 16 enters the distal portion 26 via the first gas inlet 15 at a constant rate controlled by the first control valve, and the second gas 18 enters the proximal portion 27 of the inspiratory tube 11 via the second gas inlet 17 at a constant rate, suitably controlled by a second control valve. At the start of patient exhalation, or during a pause between inhalation ending and exhalation starting, the first non-return valve 19 closes and the second gas 18 back fills the gas passageway of the inspiratory tube 11 in a direction from the proximal portion 27 toward the distal portion 26, that is away from the user. During patient exhalation, the second gas 18 and the first gas 16 forms a gas/gas interface that moves along the gas passageway away from the second gas inlet 17 toward the distal portion 26, thereby storing a volume of the second gas 18 in the inspiratory tube 11 during patient exhalation. The volume of the second gas 18, such as oxygen, that enters the inspiratory tube 11 during exhalation may be equal to, or less than, a tidal volume of the patient, thereby minimizing wastage of the second gas 18 by avoiding venting the first gas 16 during exhalation.

During patient inhalation, the first non-return valve 19 opens and the user initially receives a dose of the second gas 18 that was loaded into the inspiratory tube 11 during exhalation and is drawn into the alveoli of the user's lungs. In addition, the first gas 16 supplied into the distal portion 26 of the inspiratory tube 11 flows in a direction toward the patient, and the second gas 18 continues to be supplied to the proximal portion 27 of the inspiratory tube 11. In the event that the volume of the second gas 18 loaded into the inspiratory tube 11 is less than the tidal volume, a mixture of the first and second gases 16 and 18 can be supplied and received by the user. The first and second gases 16 and 18 supplied to the patient toward the end of the inhalation cycle is usually received within the upper regions of the inspiratory passage where gas transfer to the patient does not occur.

The pressure regulation device 22 also includes a third pressure relief device including an overpressure valve 39 for venting breathing gases from the breathing circuit 10 in the event of a blockage or malfunction. The overpressure valve 39 has a setting that is greater than the operating pressure of the first and second pressure relief devices and is located downstream of the second pressure relief valve.

Moreover, the breathing circuits 10 shown in any one of the Figures of the present specification may include the overpressure valve 39. The overpressure valve 39 is ideally located in the inspiratory tube 11, but it may also be located on the patient interface or even on the expiratory tube 12.

Set out below in Table 2 are exemplary volumes and tidal volumes of adult, paediatric and neonatal patients.

TABLE 2

| | | Example Inspiratory member internal |
|---|---|---|
| | | dimensions |
| | Typical tidal | Dia × length |
| | volume | (volume) |
| Adult | >300 mL, | 22 mm × 1.5 m |
| | typical 500 mL | (570 mL) |
| Paediatric | >50 mL and | 15 mm × 1.5 m |
| | <300 mL | (265 mL) |
| Neonatal | >10 ml and | 10 mm × 1.5 m |
| | <50 mL | (118 mL) |

As described above the inspiratory member, includes an inspiratory tube and other components such as reservoir, humidification device and so forth. Similarly, the expiratory member includes an expiratory tube and other components such as bacterial filters and so forth. The inspiratory tube and the expiratory tube may include tubing of any suitable structure. For example, the inspiratory tube and the expiratory tube may be separate tubes. In one example, the tubes may be unconnected along their length, or they may be connected side-by-side using connector clips, a permanent adhesive, or be integrally formed. An integrally formed structure may be extruded.

In another example, the inspiratory and the expiratory tubes may be provided at least in part by a multi-lumen tube, in which separate lumens provide passageways of the inspiratory and the expiratory tubes. For example, the structure of a multi-lumen tube may have side-by-side passageways, in which a partition along the tube defines in part the passageways of the inspiratory and the expiratory tubes. The multi-lumen tube may have side-by-side passageways, which has for example, been made by extrusion. In another example, the structure of the multi-lumen tube may be a coaxial structure, in which one passageway is arranged centrally, and the other passageway is arranged about the periphery of the central passageway, such as in a co-axial structure.

In yet another example, the inspiratory and the expiratory tubes may be arranged as a single conduit formed from a spirally wound hollow body. The conduit may comprise a first elongate member having a hollow body spirally wound to form at least in part an elongate tube having a hollow wall surrounding the conduit lumen. The conduit may also include a second elongate member spirally wound and joined between adjacent turns of the first elongate member. The spirally wound hollow body may provide either one of the inspiratory and the expiratory tubes, and the conduit lumen formed by the spirally wound hollow body provides the other tube.

Alternatively, the conduit lumen may be the inspiratory tube and the second gas inlet may be provided into the spirally wound hollow body. The second gas enters a distal portion of the spirally wound hollow body, and flows into a proximal portion of the conduit lumen. This allows a proximal second gas inlet without needing an additional conduit near the patient interface.

Examples of suitable spiral wound hollow bodies and multi lumen tubes are disclosed in International patent publication WO2012/164667 (PCT/IB2012/001786) filed 30 May 2012, the full contents of which are hereby incorporated into this specification.

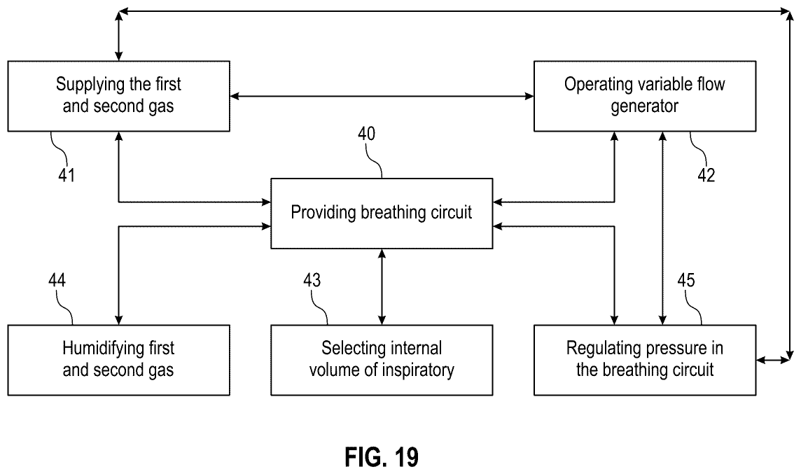

With reference to FIG. 19, a method for ventilating a patient includes providing 40 a breathing circuit 10. The breathing circuit 10 may include any one or a combination of the features of the breathing circuit 10 described herein. The method includes connecting a first gas source 13 to a distal portion 26 of the inspiratory tube 11, a second gas source 14 to inspiratory tube 11 and supplying 41 the first and second gases 16 and 18 to the inspiratory tube 11. The second gas source 14 can be connected to a distal portion 26 of the inspiratory tube 11, as shown in FIGS. 1, 2, 3, 4, 5, 6, 8, 10, 12, 13, 14 15 and 16. Alternatively, the second gas source 14 can be connected to a proximal portion 27 of the inspiratory tube 11, as shown in FIGS. 7, 9, 11 and 17.

The first and second gases 16 and 18 may be supplied at a constant rate, as shown in FIGS. 1, 2, 3, 4, 5 and 17. Alternatively, the first gas 16 can be supplied at a variable rate, according to optimal inhalation and exhalation pressures, IPAP and EPAP during bi-level therapy. A flow generator used to supply IPAP and an EPAP can also be operated to provide CPAP. The supplying 41 the first gas 16 may include operating 42 a variable flow generator 33, as shown in FIGS. 6 to 16.

In order to provide the best therapeutic benefit, it is desirable to optimize the amount of the therapeutic gas being delivered to the patient whilst minimizing wastage. This can be achieved by selecting 43 the internal volume of the inspiratory tube 11 based on the tidal volume of the patient, and changing the internal volume by changing the length of the tube 11 in which the second gas 18 is stored between inhalation cycles.

Humidification of the breathing gas can be carried out by humidifying 44 one or both of the first gas and the second gas 16 and 18 downstream of the second gas 18 entering the inspiratory tube 11.

Finally, irrespective of whether the first gas 16 is supplied at a fixed rate or at a variable rate, the method may also include regulating 45 the pressure in the breathing circuit 10. For example, the expiratory tube may include a pressure relief valve, such as PEEP valve as shown in FIGS. 1, 2, 3, 4, 7, 8, 11, 12 and 14. FIGS. 6, 9, 10 and 15 are examples in which the pressure regulation device 22 of the expiratory tube 12 include a flow restrictor 38 such as a fixed orifice, or a constant flow device. As described above, FIG. 16 is an example in which the pressure regulation device 22 includes a second flow generator 33B, suitably a variable flow generator on the expiratory tube that generates (back pressure) in the expiratory tube. The flow generator 33 allows exhaled gas to pass through the generator 33 in an opposite direction to the direction of flow being generated by the flow generator 33.

FIGS. 1, 2, 3, 4, 7, 8, 11, 12 and 14 also illustrate the pressure regulation device 22 including a pressure relief valve, such as PEEP valve for venting excess first gas supplied to the circuit.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or methods illustrated can be made without departing from the spirit of the disclosure. As will be recognized, certain embodiments of the inventions described herein can be embodied within a form that does not provide all of the features and benefits set forth herein, as some features can be used or practiced separately from others.

Reference Numeral Table

| | | | |
|---|---|---|---|
| breathing circuit | 10 | providing or obtaining | 40 |
| | | breathing circuit | |
| inspiratory tube | 11 | supplying first gas and | 41 |
| | | second gas | |
| expiratory tube | 12 | operating variable flow | 42 |
| | | generator | |
| first gas source | 13 | selecting internal volume of | 43 |
| | | the inspiratory tube | |
| second gas source | 14 | humidifying first and second | 44 |
| | | gas | |
| first gas inlet | 15 | regulating pressure in | 45 |
| | | breathing circuit | |
| first gas | 16 | reservoir | 46 |
| second gas inlet | 17 | | |
| second gas | 18 | | |
| first non-return valve | 19 | | |
| second non-return valve | 20 | | |
| patient interface | 21 | | |
| pressure regulation device | 22 | | |
| bypass tube | 23 | | |
| distal portion of expiratory | 24 | | |
| tube | | | |
| proximal portion of expiratory | 25 | | |
| tube | | | |
| distal portion of inspiratory | 26 | | |
| tube | | | |
| proximal portion of | 27 | | |
| inspiratory tube | | | |
| exhaled gas | 28 | | |
| first pressure relief valve (first | 29 | | |
| PEEP valve) | | | |
| second pressure relief valve | 30 | | |
| (first PEEP valve) | | | |
| sensor | 31 | | |
| control valve for first gas | 32A | | |
| control valve for second gas | 32B | | |
| flow generator | 33 | | |
| first flow generator | 33A | | |
| second flow generator | 33B | | |
| flow sensor | 34 | | |
| additional flow restrictor | 35 | | |
| pressure sensor | 36 | | |
| humidification device | 37 | | |
| flow restrictor | 38 | | |
| overpressure relief valve | 39 | | |

1. A positive pressure breathing circuit for ventilating a patient, the breathing circuit including:
an inspiratory member including a proximal portion that is connectable to a patient interface for supplying a breathing gas, and a distal portion that is connectable to a first source of a pressurized first gas and a second source of a pressurized second gas, wherein the second gas enters the inspiratory member downstream to where the first gas enters the inspiratory member; and
a pressure regulation device configured to regulate pressure in the breathing circuit, including venting exhaled gas.

2. The breathing circuit according to claim 1, wherein the breathing circuit includes an expiratory member which receives the exhaled gas from the patient interface.

3. The breathing circuit according to claim 2, wherein the expiratory member includes an expiratory tube extending away from the patient interface.

4. The breathing circuit according to any one of the preceding claims, wherein the inspiratory member includes an inspiratory tube.

5. The breathing circuit according to any one of the preceding claims, wherein the inspiratory member includes a first non-return valve and the first gas enters the inspiratory member upstream of the first non-return valve and the second gas enters the inspiratory member downstream of the first non-return valve.

6. The breathing circuit according to claim 5, wherein the first non-return valve is configured to inhibit the second gas flowing upstream toward the first gas entering the inspiratory member.

7. The breathing circuit according to any one of the preceding claims, wherein the inspiratory member is configured so that a volume of the second gas can enter and flow toward the patient interface without being inhaled during patient exhalation.

8. The breathing circuit according to claim 7, wherein the internal volume of the inspiratory member for receiving the volume of the second gas can be changed by changing the length of the inspiratory member to accommodate a desired volume of the second gas during patient exhalation.

9. The breathing circuit according to any one of the preceding claims when appended to claim 2, wherein the expiratory member includes a second non-return valve to inhibit the exhaled gas from re-entering the patient interface.

10. The breathing circuit according to any one of the preceding claims, wherein the pressure regulation device is directly connected to the patient interface.

11. The breathing circuit according to any one of the preceding claims when appended to claim 2, wherein the pressure regulation device is connected to the expiratory member and vents the exhaled gas from the expiratory tube.

12. The breathing circuit according to claim 10 or 11, wherein the pressure regulation device includes a pressure relief valve for venting exhaled gas, such as a positive end expiratory pressure valve (expiratory tube PEEP valve), or a restriction orifice.

13. The breathing circuit according to any one of the preceding claims, wherein the pressure regulation device is configured to regulate pressure in the inspiratory member.

14. The breathing circuit according to claim 13, wherein the pressure regulation device is located on the inspiratory member and includes a pressure relief valve, such as a positive end expiratory pressure valve (inspiratory tube PEEP valve).

15. The breathing circuit according to any one of the preceding claims, wherein the pressure regulation device is configured to regulate pressure in the expiratory member.

16. The breathing circuit according to claim 15, wherein the pressure regulation device is located on the inspiratory member and includes a pressure relief valve, such as a positive end expiratory pressure valve (inspiratory tube PEEP valve).

17. The breathing circuit according to claim 15, wherein the pressure regulation device of the expiratory member has a higher pressure setting than the pressure regulation device of the inspiratory member.

18. The breathing circuit according to claim 17, wherein the positive end expiratory pressure valve of the expiratory member may have a pressure setting ranging from about 2.5 to 35.0 cmH₂O, about 4.5.0 to 25.0 cmH₂O, about 6.5 to 15 cmH₂O, or about 8.0 to 12.0 cmH₂O, or about 10.0 cmH₂O.

19. The breathing circuit according to any one of the preceding claims, wherein the pressure regulation device includes a first control valve for controlling the pressure of the first gas supplied to the breathing circuit at a first gas inlet.

20. The breathing circuit according to any one of the preceding claims, wherein the pressure regulation device includes a second control valve for controlling the pressure of the second gas supplied to the breathing circuit at a second gas inlet.

21. The breathing circuit according to any one of the preceding claims when appended to claim 2, wherein the inspiratory member is connectable to the expiratory member so that any excess of the first gas supplied to the inspiratory member passes (from the inspiratory member) to the expiratory member without passing through the patient interface.

22. The breathing circuit according to claim 21, wherein the inspiratory member and the expiratory member are connected in a loop configuration and the excess supply of the first gas is conveyed from the inspiratory tube to the expiratory tube in the loop configuration remote from the patent interface.

23. The breathing circuit according to claim 21 or 22, wherein the distal portion of the expiratory member and the distal portion of the inspiratory member are connected to allow the excess supply of the first gas to flow from the inspiratory tube to the expiratory tube.

24. The breathing circuit according to any one of claims 21 to 23, wherein the expiratory member includes a second non-return valve to inhibit the excess supply of the first gas from entering the patient interface from the expiratory member.

25. The breathing circuit according to any one of claims 21 to 24, wherein the expiratory member is configured so that the excess supply of the first gas in the expiratory member downstream of the second non-return valve and the exhaled gas in the expiratory member downstream of the second non-return valve are vented from the breathing circuit.

26. The breathing circuit according to any one of claims 21 to 25, wherein the second non-return valve also inhibits the exhaled gas from being rebreathed during patient inhalation.

27. The breathing circuit according to any one of claims 21 to 26, wherein the breathing circuit includes a bypass member interconnecting the inspiratory member and the expiratory member that conveys the excess supply of the first gas from the inspiratory member to the expiratory member.

28. The breathing circuit according to any one of claims 21 to 27, wherein the bypass member connects to the expiratory tube downstream of the second non-return valve.

29. The breathing circuit according to any one of claims 21 to 28, wherein the bypass member includes a bypass tube.

30. The breathing circuit according to any one of claims 21 to 29, wherein the expiratory member is configured so that the excess supply of the first gas and the exhaled gas downstream of the second non-return valve are vented from the breathing circuit without re-entering the inspiratory member.

31. The breathing circuit according to any one of claims 21 to 30, wherein the pressure regulation device includes a positive end expiratory pressure valve (PEEP valve) on the distal portion of the expiratory member.

32. The breathing circuit according to claim 31, wherein the positive end expiratory pressure valve of the expiratory member may have a pressure setting ranging from about 2.5 to 35.0 cmH₂O, about 4.5.0 to 25.0 cmH₂O, about 6.5 to 15 cmH₂O, or about 8.0 to 12.0 cmH₂O, or about 10.0 cmH₂O.

33. The breathing circuit according to any one of the preceding claims, wherein the pressure regulation device includes an over pressure relief valve that is intended to release the pressure from the breathing circuit.

34. The breathing circuit according to any one of the preceding claims, wherein the breathing circuit include a humidification device for humidifying part of, or all of, the breathing gas.

35. The breathing circuit according to any one of claims 1 to 33, wherein the breathing circuit has a humidification device for humidifying the first gas, in which the humidification device is located upstream of the second gas entering the inspiratory member.

36. The breathing circuit according to any one of claims 1 to 33, wherein the breathing circuit has a humidification device for humidifying the first gas and the second gas, in which the humidification device is located downstream of the second gas entering the inspiratory tube.

37. The breathing circuit according to any one of the preceding claims, wherein the second gas is supplied a constant flow rate.

38. The breathing circuit according to any one of the preceding claims, wherein the first gas is supplied a constant flow rate.

39. The breathing circuit according to any one of the preceding claims, wherein the breathing circuit includes a flow generator for supplying the first gas to the inspiratory member.

40. The breathing circuit according to any one of the preceding claims, wherein the breathing circuit includes a variable flow generator for supplying the first gas, in which the variable flow generator is operable for supplying a high pressure during patient inhalation and a low pressure during patient exhalation for the first gas.

41. The breathing circuit according to claim 39 or 40, wherein the breathing circuit includes a sensor for detecting when a patient inhales and/or exhales, and the output of the sensor is used to operate the flow generator.

42. The breathing circuit according to claim 41, wherein the sensor includes a flow sensor for detecting the flow of the first gas in the breathing circuit, the flow sensor is located either upstream or downstream of the flow generator in the inspiratory member.

43. The breathing circuit according to claim 41, wherein the flow sensor is located in the expiratory tube to detect when the patient exhales.

44. The breathing circuit according to claim 41, wherein the sensor includes a pressure sensor for detecting the pressure of the first gas in the breathing circuit downstream of the flow generator.

45. The breathing circuit according to any one of claims 40 to 44 when appended to claim 21, wherein when the breathing circuit includes a variable flow generator, the breathing circuit has a flow restrictor restricting flow of the first gas from the inspiratory member to the expiratory member, in which a pressure drop across the flow restrictor facilitates the first gas flowing along the inspiratory tube during patient inhalation.

46. The breathing circuit according to any one of claims 1 to 20, wherein the distal portions of the inspiratory and expiratory members are unconnected, and the breathing circuit has a variable flow generator for supplying the first gas, in which the variable flow generator includes two blowers in which a first blower has an outlet connected to a distal portion of the inspiratory member, and a second blower has an outlet connected to a distal portion of the expiratory member.

47. The breathing circuit according to claim 46, wherein the second blower is operable to provide variable pressure so that exhaled gas can be vented from the expiratory member by passing through the second blower in counter flow to the direction of the pressure generated by the second blower.

48. The breathing circuit according to claim 46, wherein the second blower is operable to provide variable pressure so that exhaled gas is vented from the proximal portion of the exhalation member.

49. The breathing circuit according to any one of claims 46 to 48, wherein the first blower is operable to control the pressure at the patient interface when the patient inhales.

50. The breathing circuit according to any one of claims 46 to 49, wherein during patient exhalation, the first blower is operatable at a lower pressure, or at a lower flow than during patient inhalation.

51. The breathing circuit according to any one of claims 46 to 50, wherein during patient exhalation, the first blower is operatable at a lower pressure or lower flow than the pressure or flow setting of the second blower.

52. The breathing circuit according to any one of claims 46 to 51, wherein during patient inhalation, the first blower may be operated at a higher pressure, or higher flow than the pressure or flow setting of the second blower.

53. The breathing circuit according to any one of the preceding claims, wherein the inspiratory tube has a length ranging from about 0.5 m to 2.5 m, or about a length ranging from 0.75 to 2.0 m, or a length ranging from about 1.5 to 1.8 m.

54. The breathing circuit according to any one of the preceding claims, wherein the volume of the first gas that may enter the inspiratory tube during patient exhalation may range from about 50 to 100 percent by volume of a tidal volume of a patient, or from about 50 to 90 percent by volume of a tidal volume of a patient, or range from about 60 to 70 percent by volume of a tidal volume of a patient.

55. The breathing circuit according to any one of the preceding claims, wherein the inspiratory member has an internal volume: i) for adult patients, ranging from about 315 ml to 760 ml, or from about 400 to 600 ml; ii) for pediatric patients, ranging from about 100 ml to 450 ml, or range from about 200 to 400 ml; and iii) for neonatal patients, ranging from about 50 to 200 ml, or range from about 100 to 150 ml.

56. The breathing circuit according to any one of the preceding claims, wherein the first gas is pressurized air.

57. The breathing circuit according to any one of the preceding claims, wherein the second gas is pressurized oxygen gas.

58. The breathing circuit according to any one of the preceding claims, wherein the second gas is a pressurized gas including one or any combination of: oxygen gas, heliox, or an anaesthetic gas. The anaesthetic gas could be nitrous oxide or a 50:50 mixture of nitrous oxide and oxygen gas.

59. The breathing circuit according to any one of the preceding claims, wherein the breathing circuit includes a sealed patient interface.

60. A positive pressure breathing circuit for ventilating a patient, the breathing circuit including:
an inspiratory member including a proximal portion that is connectable to a patient interface for supplying a breathing gas, and a distal portion that is connectable to a source of a pressurized first gas, wherein the inspiratory member is connectable to a pressurized second gas that enters the inspiratory member downstream of a position to where the first gas enters the inspiratory member; and
a flow generator for supplying the first gas at a controlled pressure to the inspiratory member.

61. The breathing circuit according to claim 60, wherein the breathing circuit includes a pressure regulation device configured to regulate pressure in the breathing circuit.

62. The breathing circuit according to claim 60 or 61, wherein the breathing circuit includes an expiratory member which is configured to receive the exhaled gas from the patient interface and vent the exhaled gas.

63. The breathing circuit according to claim 62, wherein the expiratory member includes an expiratory tube extending away from the patient interface.

64. The breathing circuit according to any one of claims 60 to 63, wherein the inspiratory member includes an inspiratory tube.

65. The breathing circuit according to any one of claims 60 to 64, wherein the inspiratory member includes a first non-return valve and the first gas enters the inspiratory member upstream of the first non-return valve and the second gas enters the inspiratory member downstream of the first non-return valve.

66. The breathing circuit according to claim 65, wherein the first non-return valve is configured to inhibit the second gas flowing upstream toward the first gas entering the inspiratory member.

67. The breathing circuit according to any one of claims 60 to 66, wherein the inspiratory member is configured so that a volume of the second gas can enter and flow toward the patient interface without being inhaled during patient exhalation.

68. The breathing circuit according to claim 67, wherein the internal volume of the inspiratory member for receiving the volume of the second gas can be changed by changing the length of the inspiratory member to accommodate a desired volume of the second gas during patient exhalation.

69. The breathing circuit according to any one of claims 60 to 68, wherein the expiratory member includes a second non-return valve to inhibit the exhaled gas from re-entering the patient interface.

70. The breathing circuit according to any one of claims 62 to 69 when appended to claim 61, wherein the pressure regulation device is directly connected to the patient interface.

71. The breathing circuit according to any one of claims 62 to 70 when appended to claim 61, wherein the pressure regulation device is connected to the expiratory member and vents the exhaled gas from the expiratory tube.

72. The breathing circuit according to claim 70 or 71, wherein the pressure regulation device includes a pressure relief valve for venting exhaled gas, such as a positive end expiratory pressure valve (expiratory tube PEEP valve), or a restriction orifice.

73. The breathing circuit according to any one of claims 60 to 72, wherein the pressure regulation device is configured to regulate pressure in the inspiratory member.

74. The breathing circuit according to claim 73, wherein the pressure regulation device is located on the inspiratory member includes a pressure relief valve, such as a positive end expiratory pressure valve (inspiratory tube PEEP valve).

75. The breathing circuit according to any one of 62 to 74 when appended to claim 61, wherein the pressure regulation device is configured to regulate pressure in the expiratory member.

76. The breathing circuit according to claim 75, wherein the pressure regulation device is located on the inspiratory member and includes a pressure relief valve, such as a positive end expiratory pressure valve (inspiratory tube PEEP valve).

77. The breathing circuit according to claim 75, wherein the pressure regulation device of the expiratory member has a higher pressure setting than the pressure regulation device of the inspiratory member.

78. The breathing circuit according to claim 77, wherein the positive end expiratory pressure valve of the expiratory member may have a pressure setting ranging from about 2.5 to 35.0 cmH₂O, about 4.5.0 to 25.0 cmH₂O, about 6.5 to 15 cmH₂O, or about 8.0 to 12.0 cmH₂O, or about 10.0 cmH₂O.

79. The breathing circuit according to any one of claims 60 to 78, wherein the pressure regulation device includes a first control valve for controlling the pressure of the first gas supplied to the breathing circuit at a first gas inlet.

80. The breathing circuit according to any one of claims 60 to 79, wherein the pressure regulation device includes a second control valve for controlling the pressure of the second gas supplied to the breathing circuit at a second gas inlet.

81. The breathing circuit according to any one of claims 62 to 80 when appended to claim 61, wherein the inspiratory member is connectable to the expiratory member so that any excess of the first gas supplied to the inspiratory member passes (from the inspiratory member) to the expiratory member without passing through the patient interface.

82. The breathing circuit according to claim 81, wherein the inspiratory member and the expiratory member are connected in a loop configuration and the excess supply of the first gas is conveyed from the inspiratory tube to the expiratory tube in the loop configuration remote from the patent interface.

83. The breathing circuit according to claim 81 or 82, wherein the distal portion of the expiratory member and the distal portion of the inspiratory member are connected to allow the excess supply of the first gas to flow from the inspiratory tube to the expiratory tube.

84. The breathing circuit according to any one of claims 81 to 82, wherein the expiratory member includes a second non-return valve to inhibit the excess supply of the first gas from entering the patient interface from the expiratory member.

85. The breathing circuit according to any one of claims 81 to 84, wherein the expiratory member is configured so that the excess supply of the first gas in the expiratory member downstream of the second non-return valve and the exhaled gas in the expiratory member downstream of the second non-return valve are vented from the breathing circuit.

86. The breathing circuit according to any one of claims 81 to 85, wherein the second non-return valve also inhibits the exhaled gas from being rebreathed during patient inhalation.

87. The breathing circuit according to any one of claims 81 to 86, wherein the breathing circuit includes a bypass member interconnecting the inspiratory member and the expiratory member that conveys the excess supply of the first gas from the inspiratory member to the expiratory member.

88. The breathing circuit according to any one of claims 81 to 87, wherein the bypass member connects to the expiratory tube downstream of the second non-return valve.

89. The breathing circuit according to any one of claims 81 to 88, wherein the bypass member includes a bypass tube.

90. The breathing circuit according to any one of claims 81 to 89, wherein the expiratory member is configured so that the excess supply of the first gas and the exhaled gas downstream of the second non-return valve are vented from the breathing circuit without re-entering the inspiratory member.

91. The breathing circuit according to any one of claims 81 to 90, wherein the pressure regulation device includes a positive end expiratory pressure valve (PEEP valve) on the distal portion of the expiratory member.

92. The breathing circuit according to claim 91, wherein the positive end expiratory pressure valve of the expiratory member may have a pressure setting ranging from about 2.5 to 35.0 cmH₂O, about 4.5.0 to 25.0 cmH₂O, about 6.5 to 15 cmH₂O, or about 8.0 to 12.0 cmH₂O, or about 10.0 cmH₂O.

93. The breathing circuit according to any one of claims 60 to 92, wherein the pressure regulation device includes an over pressure relief valve that is intended to release the pressure from the breathing circuit.

94. The breathing circuit according to any one of claims 60 to 93, wherein the breathing circuit include a humidification device for humidifying part of, or all of, the breathing gas.

95. The breathing circuit according to any one of claims 60 to 93, wherein the breathing circuit has a humidification device for humidifying the first gas, in which the humidification device is located upstream of the second gas entering the inspiratory member.

96. The breathing circuit according to any one of claims 60 to 93, wherein the breathing circuit has a humidification device for humidifying the first gas and the second gas, in which the humidification device is located downstream of the second gas entering the inspiratory tube.

97. The breathing circuit according to any one of claims 60 to 96, wherein the second gas is supplied a constant flow rate.

98. The breathing circuit according to any one of claims 60 to 97, wherein the first gas is supplied a constant flow rate.

99. The breathing circuit according to any one of claims 60 to 98, wherein the breathing circuit includes a flow generator for supplying the first gas to the inspiratory member.

100. The breathing circuit according to any one of claims 60 to 99, wherein the breathing circuit includes a variable flow generator for supplying the first gas, in which the variable flow generator is operable for supplying a high pressure during patient inhalation and a low pressure during patient exhalation for the first gas.

101. The breathing circuit according to claim 79 or 100, wherein the breathing circuit includes a sensor for detecting when a patient inhales and/or exhales, and the output of the sensor is used to operate the flow generator.

102. The breathing circuit according to claim 101, wherein the sensor includes a flow sensor for detecting the flow of the first gas in the breathing circuit, the flow sensor is located either upstream or downstream of the flow generator in the inspiratory member.

103. The breathing circuit according to claim 101, wherein the flow sensor is located in the expiratory tube to detect when the patient exhales.

104. The breathing circuit according to claim 101, wherein the sensor includes a pressure sensor for detecting the pressure of the first gas in the breathing circuit downstream of the flow generator.

105. The breathing circuit according to any one of claims 100 to 104 when appended to claim 81, wherein when the breathing circuit includes a variable flow generator, the breathing circuit has a flow restrictor restricting flow of the first gas from the inspiratory member to the expiratory member, in which a pressure drop across the flow restrictor facilitates the first gas flowing along the inspiratory tube during patient inhalation.

106. The breathing circuit according to any one of claims 60 to 80, wherein the distal portions of the inspiratory and expiratory members are unconnected, and the breathing circuit has a variable flow generator for supplying the first gas, in which the variable flow generator includes two blowers in which a first blower has an outlet connected to a distal portion of the inspiratory member, and a second blower has an outlet connected to a distal portion of the expiratory member.

107. The breathing circuit according to claim 106, wherein the second blower is operable to provide variable pressure so that exhaled gas can be vented from the expiratory member by passing through the second blower in counter flow to the direction of the pressure generated by the second blower.

108. The breathing circuit according to claim 106, wherein the second blower is operable to provide variable pressure so that exhaled gas is vented from the proximal portion of the exhalation member.

109. The breathing circuit according to any one of claims 106 to 108, wherein the first blower is operable to control the pressure at the patient interface when the patient inhales.

110. The breathing circuit according to any one of claims 106 to 109, wherein during patient exhalation, the first blower is operatable at a lower pressure, or at a lower flow than during patient inhalation.

111. The breathing circuit according to any one of claims 106 to 110, wherein during patient exhalation, the first blower is operatable at a lower pressure or lower flow than the pressure or flow setting of the second blower.

112. The breathing circuit according to any one of claims 106 to 111, wherein during patient inhalation, the first blower may be operated at a higher pressure, or higher flow than the pressure or flow setting of the second blower.

113. The breathing circuit according to any one of claims 60 to 112, wherein the inspiratory tube has a length ranging from about 0.5 m to 2.5 m, or about a length ranging from 0.75 to 2.0 m, or a length ranging from about 1.5 to 1.8 m.

114. The breathing circuit according to any one of claims 60 to 113, wherein the volume of the first gas that may enter the inspiratory tube during patient exhalation may range from about 50 to 100 percent by volume of a tidal volume of a patient, or from about 50 to 90 percent by volume of a tidal volume of a patient, or range from about 60 to 70 percent by volume of a tidal volume of a patient.

115. The breathing circuit according to any one of claims 60 to 114, wherein the inspiratory member has an internal volume: i) for adult patients, ranging from about 315 ml to 760 ml, or from about 400 to 600 ml; ii) for pediatric patients, ranging from about 100 ml to 450 ml, or range from about 200 to 400 ml; and iii) for neonatal patients, ranging from about 50 to 200 ml, or range from about 100 to 150 ml.

116. The breathing circuit according to any one of claims 60 to 115, wherein the first gas is pressurized air.

117. The breathing circuit according to any one of claims 60 to 116, wherein the second gas is pressurized oxygen gas.

118. The breathing circuit according to any one of claims 60 to 117, wherein the second gas is a pressurized gas including one or any combination of: oxygen gas, heliox, or an anaesthetic gas. The anaesthetic gas could be nitrous oxide or a 50:50 mixture of nitrous oxide and oxygen gas.

119. The breathing circuit according to any one of claims 60 to 118, wherein the breathing circuit includes a sealed patient interface.

120. A method for ventilating a patient, the method including:
providing a positive pressure breathing circuit having:
an inspiratory member including a proximal portion that is connectable to a patient interface for supplying a breathing gas, and a distal portion that is connectable to a first source of a pressurized first gas and a second source of a pressurized second gas; and
a pressure regulation device configurated to regulate pressure in the breathing circuit, including venting exhaled gas; and
supplying the first gas and the second gas into the distal portion of the inspiratory member, wherein the second gas enters the inspiratory member downstream to where the first gas enters the inspiratory member.

121. The method according to claim 120, wherein the step of providing the breathing circuit includes an expiratory member configured to receive exhaled gas from the patient interface.

122. The method according to claim 121, wherein the method includes a step of regulating the pressure in the breathing circuit which includes venting exhaled gas from the expiratory member.

123. The method according to claims 121 or 122, wherein the distal portions of the inspiratory member and the expiratory member are interconnected to form a loop configuration, and the method includes the first gas being supplied to the inspiratory member and any excess supply of the first gas is conveyed from the inspiratory member to the expiratory member by the interconnection of the inspiratory member and the expiratory member without passing through the patient interface.

124. The method according to claim 123, wherein the breathing circuit provided may be configured with a bypass member interconnecting the inspiratory member and the expiratory member.

125. The method according to claim 124, wherein the step of regulating the pressure includes conveying the excess supply of the first gas from the inspiratory member to the expiratory member via the bypass member.

126. The method according to any one of claims 120 to 125, wherein the method includes humidifying the breathing gas.

127. The method according to any one of claims 120 to 126, wherein the second gas is supplied at a substantially constant flow rate.

128. The method according to any one of claims 120 to 127, wherein the first gas may be supplied at a constant flow rate.

129. The method according to any one of claims 120 to 127, wherein the method includes operating a variable flow generator to supply the first gas at a variable flow rate.

130. The method according to claims 129, wherein the step of operating the variable flow generator includes sensing flow in the breathing circuit and using output data of a sensor sensing the flow to operate the variable flow generator.

131. The method according to claims 130, wherein the output data of the sensor correspond to when the patient inhales and/or exhales.

132. The method according to claim 121, wherein the method includes selecting an internal volume of the inspiratory member in which the second gas can be stored.

133. The method according to claim 129, wherein when the distal portions of the inspiratory and expiratory members are unconnected, the variable flow generator includes two blowers in which a first blower has an outlet connected to a distal portion of the inspiratory member, and a second blower has an outlet connected to a distal portion of the expiratory member, and the step operating the variable flow generator includes operating the second blower to provide variable pressure so that exhaled gas can be vented from the expiratory member by passing through the second blower in counter flow to the direction of the pressure generated by the second blower.

134. The method according to claim 133, wherein the method includes operating second blower to provide variable pressure so that exhaled gas is vented from the proximal portion of the exhalation member.

135. The method according to claim 133, wherein the method includes operating the first blower to control the pressure at the patient interface when the patient inhales.

136. The method according to claim 133, wherein during patient exhalation, the first blower is operated at a lower pressure, or at a lower flow, than during patient inhalation.

137. The method according to claim 133, wherein during patient inhalation, the first blower is operated at a higher pressure, or higher flow than the pressure or flow setting of the second blower.

138. A method for ventilating a patient, the method including:
providing a positive pressure breathing circuit including:
an inspiratory member including a proximal portion that is connectable to a patient interface for supplying a breathing gas, and a distal portion that is connectable to a source of a pressurized first gas, wherein the inspiratory member is connectable to a pressurized second gas that enters the inspiratory member downstream of a position to where the first gas enters the inspiratory member; and
a variable flow generator for supplying the first gas, and
operating the variable flow generator between high pressure during the patient inhaling and low pressure during the patient exhaling to regulate the pressure in the inspiratory member.

139. The method according to claim 138, wherein the breathing circuit includes an expiratory member configured to vent the exhaled gas from the patient interface, and the distal portions of the inspiratory member and the expiratory member are interconnected to form a loop configuration, and the method includes the first gas being supplied to the inspiratory member and any excess supply of the first gas is conveyed from the inspiratory member to the expiratory member by the interconnection of the inspiratory member and the expiratory member without passing through the patient interface.

140. The method according to claim 139, wherein the breathing circuit provided may be configured with a bypass member interconnecting the inspiratory member and the expiratory member.

141. The method according to claim 140, wherein the step of regulating the pressure includes conveying the excess supply of the first gas from the inspiratory member to the expiratory member via the bypass member.

142. The method according to any one of claims 138 to 141, wherein the method includes humidifying the breathing gas.

143. The method according to any one of claims 138 to 142, wherein the second gas is supplied at a substantially constant flow rate.

144. The method according to any one of claims 138 to 143, wherein the first gas may be supplied at a constant flow rate.

145. The method according to any one of claims 138 to 144, wherein the method includes operating a variable flow generator to supply the first gas at a variable flow rate.

146. The method according to claim 145, wherein the step of operating the variable flow generator includes sensing flow in the breathing circuit and using output data of a sensor sensing the flow to operate the variable flow generator.

147. The method according to claim 146, wherein the output data of the sensor correspond to when the patient inhales and/or exhales.

148. The method according to any one of claims 138 to 147, wherein the method includes selecting an internal volume of the inspiratory member in which the second gas can be stored.

149. The method according to claim 145, wherein when the distal portions of the inspiratory and expiratory members are unconnected, the variable flow generator includes two blowers in which a first blower has an outlet connected to a distal portion of the inspiratory member, and a second blower has an outlet connected to a distal portion of the expiratory member, and the step operating the variable flow generator includes operating the second blower to provide variable pressure so that exhaled gas can be vented from the expiratory member by passing through the second blower in counter flow to the direction of the pressure generated by the second blower.

150. The method according to claim 149, wherein the method includes operating second blower to provide variable pressure so that exhaled gas is vented from the proximal portion of the exhalation member.

151. The method according to claim 149, wherein the method includes operating the first blower to control the pressure at the patient interface when the patient inhales.

152. The method according to claim 149, wherein during patient exhalation, the first blower is operated at a lower pressure, or at a lower flow, than during patient inhalation.

153. The method according to claim 149, wherein during patient inhalation, the first blower is operated at a higher pressure, or higher flow than the pressure or flow setting of the second blower.
